(19) United States
(12) Patent Application Publication
(10) Pub. No.: US 2022/0364059 A1
(43) Pub. Date: Nov. 17, 2022

(54) PRIMING MEDIA AND METHODS FOR STEM CELL CULTURE AND THERAPY

(71) Applicant: Case Western Reserve University, Cleveland, OH (US)

(72) Inventors: Hulya BUKULMEZ, Cleveland, OH (US); Arnold CAPLAN, Cleveland, OH (US)

(21) Appl. No.: 17/624,347

(22) PCT Filed: Jul. 6, 2020
(86) PCT No.: PCT/US2020/040931

§ 371 Date: Jan. 2, 2022

Related U.S. Application Data
(60) Provisional application No. 62/870,832, filed on Jul. 5, 2019.
(60) Provisional application No. 62/908,762, filed on Oct. 1, 2019.

(51) Int. Cl. C12N 5/0775

(52) U.S. Cl. CPC C12N 5/0663; C12N 2500/90; C12N 2500/36; C12N 2501/25; C12N 2501/2301; C12N 2501/2317; C12N 2501/24

(57) ABSTRACT

One aspect of the present disclosure can include a priming medium for creating an isolated population of stem cells having an anti-inflammatory phenotype from an unprimed population of stem cells. The priming media can include a serum-free medium, a functional activator of a Type I interferon (IFN) pathway and a Type II IFN pathway, and at least two pro-inflammatory cytokines. The functional activator and the at least two pro-inflammatory cytokines can be present in an amount sufficient to promote induction of stem cells having an anti-inflammatory phenotype. The cells having an anti-inflammatory phenotype can be marked by increased expression and/or secretion of one or more anti-inflammatory or immune modulatory mediators as compared to the unprimed population of stem cells. Other aspects of the present disclosure can include stem cells made according to the present disclosure as well as therapeutic compositions and uses of the stem cells.

### RELATED APPLICATIONS

This application claims priority to U.S. Provisional Patent Application Ser. Nos. 62/870,832, filed Jul. 5, 2019, and 62/908,762, filed Oct. 1, 2019. The entirety of each of the aforementioned U.S. Provisional Patent Applications is hereby incorporated by reference for all purposes.

### TECHNICAL FIELD

The present disclosure is generally directed to priming media and methods for stem cell culture and therapy and, more specifically, to novel stem cell culture and therapy methods and culture media compositions for the purpose of inducing, activating, or priming discrete uniform cell phenotypes to selectively suppress inflammation and promote immunity, providing significant advantages over known culture media and methods, yielding primed, activated, or induced stem cells used in cell-based therapy.

### BACKGROUND

Clinical applications of stem cells (e.g., mesenchymal stem cells or MSCs) require reproducible cell culture methods and cell expansion methods that provide adequate numbers of cells of suitable quality and consistent therapeutic benefits. Different culture media and methods have had varying degrees of success. There remains a need for further improvements to stem cell culture media and methods that ensures expanded yields of primed, activated, or induced cells used in cell-based therapy having safe and consistently reproducible therapeutic effects.

### SUMMARY

Aspects of the present disclosure can be used to provide more uniform and predictable ex vivo expanded and induced, primed, or activated populations of stem cells (e.g., mesenchymal stem cells or MSCs), which can be used for cell-based therapy. There is a long-felt need in the art for an improved method to provide a uniform and efficacious large number of stem cells required for cell-based therapy. An advantage of the various aspects of the present disclosure is that stem cells produced by the priming media and methods disclosed herein can be used to induce, activate or prime cultures of stem cells into uniform and discrete anti-inflammatory phenotypes that behave in a predictable manner upon introduction into a subject.

According to one aspect of the present disclosure, there is a need for improved therapeutic methods and improved cell culture methods and media for inducing, activating, or priming uniform populations of MSCs derived from various adult tissues. The potency or therapeutic benefit of induced, activated, or primed MSC over un-induced or unprimed conventional MSCs has been demonstrated in vitro as well as in pre-clinical models of disease.

It has been surprisingly discovered that stem cells (e.g., MSCs), when exposed to priming media having a specific combination of components, are primed to specifically regulate immune cellular activity and improve immune cell dysfunction, particularly after type I and II interferon pathway induction by autoimmunity. Primed stem cells (e.g., MSCs) showed safety and efficacy in in vivo models of induced interferon activation (e.g., pristane induced systemic lupus erythematosus) mice models and its complications. Advantageously, stem cells (e.g., MSCs) exposed to priming media of the present application obtain a state or phenotype where they promote a potent anti-inflammatory response, control innate and adaptive immune cellular activity, and/or regulate interferons while producing factors that promote angiogenesis and tissue regeneration. Without being bound by theory, it is believed that primed stem cells (e.g., MSCs) of the present application exhibit these potent immune regulatory effects by expanding and activating CD4+ T cells and CD8+ T-cells, NK cells and T regulatory (Treg) cells, while suppressing dendritic cells (DC) and B-cells by controlling the production and activity of Type I (-α, -β) and II interferons (-γ) and their downstream effectors, response to molecules, and other inflammatory signaling pathways.

Based at least in part on the foregoing discoveries, one aspect of the present disclosure can include a priming medium for creating an isolated population of stem cells having an anti-inflammatory phenotype from an unprimed population of stem cells. The priming media can comprise a serum-free medium, a functional activator of a Type I interferon (IFN) pathway and a Type II IFN pathway, and at least two pro-inflammatory cytokines. The functional activator and the at least two pro-inflammatory cytokines can be present in an amount sufficient to promote induction of stem cells having an anti-inflammatory phenotype. The cells having an anti-inflammatory phenotype can be marked by increased expression and/or secretion of one or more anti-inflammatory or immune modulatory mediators as compared to the unprimed population of stem cells.

Another aspect of the present disclosure can include a composition comprising a priming medium and stem cells. The priming medium can comprise a serum-free medium, a functional activator of a Type I IFN pathway and a Type II IFN pathway, and at least two pro-inflammatory cytokines. The functional activator and the at least two pro-inflammatory cytokines can be present in an amount sufficient to promote induction of stem cells having an anti-inflammatory phenotype. The cells having an anti-inflammatory phenotype can be marked by increased expression and/or secretion of one or more anti-inflammatory or immune modulatory mediators as compared to the unprimed population of stem cells.

Another aspect of the present disclosure can include an in vitro method for creating an isolated population of stem cells having an anti-inflammatory phenotype. The method can include contacting an unprimed population of stem cells with a priming medium for a time and under conditions sufficient to promote induction of stem cells having an anti-inflammatory phenotype. The priming medium can comprise a serum-free medium, a functional activator of a Type I IFN pathway and a Type II IFN pathway, and at least two pro-inflammatory cytokines. The cells can have an anti-inflammatory phenotype are marked by increased expression of one or more anti-inflammatory or immune modulatory mediators as compared to unprimed stem cells.

Another aspect of the present disclosure can include an isolated population of stem cells having an anti-inflammatory phenotype created by an in vitro method. The method can include contacting an unprimed population of stem cells with a priming medium for a time and under conditions sufficient to promote induction of stem cells having an anti-inflammatory phenotype. The priming medium can comprise a serum-free medium, a functional activator of a Type I IFN pathway and a Type II IFN pathway, and at least two pro-inflammatory cytokines. The cells that can have an anti-inflammatory phenotype are marked by increased expression of one or more anti-inflammatory or immune modulatory mediators as compared to unprimed stem cells.

Stem cells created by an in vitro method of the present disclosure are markedly different than naturally occurring stem cells. This is because level or degree of anti-inflammatory gene expression (e.g., indoleamine 2,3-dioxygenase or IDO) is significantly higher than what would occur in nature (e.g., 50,000 to 150,000 times greater in the stem cells of the present disclosure as compared to stem cells found in nature). The same is true for the level or degree of surface marker expression in primed stem cells (e.g., MSCs) created by an in vitro method of the present disclosure where, for example, CD146 is expressed at significantly higher levels than what would occur in nature, giving them a pericyte-like phenotype. Further, stem cells created by a method of the present disclosure are markedly different than naturally-occurring stem cells because of the structural and functional differences that result in the primed stem cells (of the present disclosure) as a result of exposure to the priming media of the present disclosure, which contains a unique combination of components in serum-free media (and, in some instances, as a defined media), which is unlike an in vivo inflammatory state where numerous undefined molecules (e.g., proteins, carbohydrates, metabolites, etc.) are present.

Another aspect of the present disclosure can include a method for treating a subject for a systemic inflammatory disease or disorder. The method can include administering to the subject a therapeutically effective amount of stem cells having an anti-inflammatory phenotype. Prior to administration, the stem cells are created by an in vitro method. The method can include contacting an unprimed population of stem cells with a priming medium for a time and under conditions sufficient to promote induction of stem cells having an anti-inflammatory phenotype. The priming medium can comprise a serum-free medium, a functional activator of a Type I IFN pathway and a Type II IFN pathway, and at least two pro-inflammatory cytokines. The cells can have an anti-inflammatory phenotype are marked by increased expression of one or more anti-inflammatory or immune modulatory mediators as compared to unprimed stem cells.

Another aspect of the present disclosure can include a priming medium for creating an isolated population of stem cells having an anti-inflammatory phenotype from an unprimed population of stem cells. The priming medium can comprise a serum-free medium, a functional activator of a Type I IFN pathway and a Type II IFN pathway, at least four pro-inflammatory cytokines, and an essential vitamin. The functional activator and the at least four pro-inflammatory cytokines can be present in an amount sufficient to promote induction of stem cells having an anti-inflammatory phenotype. The cells having an anti-inflammatory phenotype can be marked by increased expression and/or secretion of one or more anti-inflammatory or immune modulatory mediators as compared to the unprimed population of stem cells.

Another aspect of the present disclosure can include a composition comprising a priming medium and stem cells. The priming medium can comprise a serum-free medium, a functional activator of a Type I IFN pathway and a Type II IFN pathway, at least four pro-inflammatory cytokines, and an essential vitamin. The functional activator and the at least four pro-inflammatory cytokines can be present in an amount sufficient to promote induction of stem cells having an anti-inflammatory phenotype. The cells having an anti-inflammatory phenotype can be marked by increased expression and/or secretion of one or more anti-inflammatory or immune modulatory mediators as compared to the unprimed population of stem cells.

Another aspect of the present disclosure can include an in vitro method for creating an isolated population of stem cells having an anti-inflammatory phenotype. The method can include contacting an unprimed population of stem cells with a priming medium for a time and under conditions sufficient to promote induction of stem cells having an anti-inflammatory phenotype. The priming medium can comprise a serum-free medium, a functional activator of a Type I IFN pathway and a Type II IFN pathway, at least four pro-inflammatory cytokines, and an essential vitamin. The cells can have an anti-inflammatory phenotype are marked by increased expression of one or more anti-inflammatory or immune modulatory mediators as compared to unprimed stem cells.

Another aspect of the present disclosure can include an isolated population of stem cells having an anti-inflammatory phenotype created by an in vitro method. The method can include contacting an unprimed population of stem cells with a priming medium for a time and under conditions sufficient to promote induction of stem cells having an anti-inflammatory phenotype. The priming medium can comprise a serum-free medium, a functional activator of a Type I IFN pathway and a Type II IFN pathway, at least four pro-inflammatory cytokines, and an essential vitamin. The cells can have an anti-inflammatory phenotype are marked by increased expression of one or more anti-inflammatory or immune modulatory mediators as compared to unprimed stem cells.

Another aspect of the present disclosure can include a method for treating a subject for a systemic inflammatory disease or disorder. The method can comprise administering to the subject a therapeutically effective amount of stem cells having an anti-inflammatory phenotype. Prior to administration, the stem cells are created by an in vitro method. The method can include contacting an unprimed population of stem cells with a priming medium for a time and under conditions sufficient to promote induction of stem cells having an anti-inflammatory phenotype. The priming medium can comprise a serum-free medium, a functional activator of a Type I IFN pathway and a Type II IFN pathway, at least four pro-inflammatory cytokines, and an essential vitamin. The cells that can have an anti-inflammatory phenotype are marked by increased expression of one or more anti-inflammatory or immune modulatory mediators as compared to unprimed stem cells.

Another aspect of the present disclosure can include a method for adoptive immunotherapy in a subject. The method can comprise exposing unprimed CAR-T cells and/or CAR-NK cells for a time and under conditions sufficient to prime and expand the CAR-T cells and/or CAR-NK cells and increase the cytotoxic activity of the CAR-T cells and/or CAR-NK cells. The primed CAR-T cells and/or CAR-NK cells can be created by an in vitro method of the present disclosure. The in vitro method can include contacting an unprimed population of CAR-T cells and/or CAR-NK cells with a priming medium for a time and under conditions sufficient to prime and expand the CAR-T cells and/or CAR-NK cells and increase the cytotoxic activity of the CAR-T cells and/or CAR-NK cells as compared to unprimed CAR-T cells and/or CAR-NK cells. In some instances, the priming medium can comprise a serum-free medium, a functional activator of a Type I IFN pathway and a Type II IFN pathway, and at least two pro-inflammatory cytokines. In other instances, the priming medium can comprise a serum-free medium, a functional activator of a Type I IFN pathway and a Type II IFN pathway, at least four pro-inflammatory cytokines, and an essential vitamin A therapeutically effective dose of the primed CAR-T cells and/or CAR-NK cells can be administered to a subject in need of adoptive immunotherapy.

Another aspect of the present disclosure can include a method for drug discovery. The method can comprise exposing, in vitro, a population of stem cells produced by an in vitro method and having an anti-inflammatory phenotype to an agent to assess the effect of the agent on the ability of the stem cells having an increased anti-inflammatory phenotype to affect inflammation. The in vitro method can include contacting an unprimed population of stem cells with a priming medium for a time and under conditions sufficient to promote induction of the stem cells having an anti-inflammatory phenotype. In some instances, the priming medium can comprise a serum-free medium, a functional activator of a Type I IFN pathway and a Type II IFN pathway, at least four pro-inflammatory cytokines, and an essential vitamin. In other instances, the priming medium can comprise a serum-free medium, a functional activator of a Type I IFN pathway and a Type II IFN pathway, and at least two pro-inflammatory cytokines. The stem cells can have an anti-inflammatory phenotype that is marked by increased expression of one or more anti-inflammatory or immune modulatory mediators as compared to unprimed stem cells.

Another aspect of the present disclosure can include use of stem cells having an anti-inflammatory phenotype produced by an in vitro method for development and study of a disease model of inflammation. The in vitro method can include contacting an unprimed population of stem cells with a priming medium for a time and under conditions sufficient to promote induction of the stem cells having an anti-inflammatory phenotype. In some instances, the priming medium can comprise a serum-free medium, a functional activator of a Type I IFN pathway and a Type II IFN pathway, at least four pro-inflammatory cytokines, and an essential vitamin. In other instances, the priming medium can comprise a serum-free medium, a functional activator of a Type I IFN pathway and a Type II IFN pathway, and at least two pro-inflammatory cytokines. The cells can have an anti-inflammatory phenotype that is marked by increased expression of one or more anti-inflammatory or immune modulatory mediators as compared to unprimed stem cells.

### BRIEF DESCRIPTIONS OF THE DRAWINGS

The foregoing and other features of the present disclosure will become apparent to those skilled in the art to which the present disclosure relates upon reading the following description with reference to the accompanying drawings, in which:

FIGS. 1A-B are schematic illustrations showing a network of cytokine pathways (red=upreglation, green=downreguation) (FIG. 1A) and a network of cellular movement pathways (FIG. 1B) after exposure of mesenchymal stem cells (MSCs) to Poly (I:C);

FIG. 2 is flow cytometery data showing that Poly (I:C) treatment increases CD146+ MSCs in mixed bone marrow isolated MSC cultures (shown are MSCs from two different healthy bone marrow donors);

FIG. 3 is a schematic illustration showing validation of the priming media of the present disclosure. MSCs were cultured using robotic cell culturing technology and with a priming medium of the present disclosure. As an efficacy assay, indoleamine 2,3-dioxygenase (IDO) gene expression fold difference was verified in primed MSCs using RT-PCR. Isolated RNA was collected from the MSCs at different time points of priming period (6 hours, 12 hours, 18 hours and 24 hours). Maximum IDO gene expression was measured across three unrelated primed MSC sources (numbered as 2183, 1427 healthy donor, at passage 3 and Rooster Bioscience, a commercial MSC product with unknown passage number). Condition 1 represents the priming medium condition while Condition 2 is a control condition;

FIG. 4 is a series of graphs showing IDO gene expression fold difference and activity levels (N-formyl-kynurenine enzyme level (mcg/ml) in the cell culture media) significantly increased after exposure to a priming medium of the present disclosure after 18 hours;

FIG. 5 is a graph showing perivascular MSC markers (PDGFBP and CD146) gene expression fold differences using RT-PCR (green=18 hours post-exposure to priming medium of the present disclosure);

FIG. 6 shows the results of peritoneal lavage fluid cell sorting in a pristane mouse model one week after treatment of primed MSCs (after treatment with a priming medium of the present disclosure for 18 hours) (n=7) and unprimed MSCs (n=6). The results indicate significant (*) increase in CD3+CD69+CD4+ T cells (blue-colored bars) and CD3+CD69+CD8+ T cells (red bars) in the treated (primed, Pristane-HXB-319) subset as compared to the unprimed subset (Pristane-MSC) and sham (Pristane-sham) untreated pristane injected mice;

FIGS. 7A-B show the results of cell sorting experiments on human peripheral blood mononuclear cells (PBMCs) (FIG. 7A) and natural killer (NK) cells (FIG. 7B). FIG. 7A shows the results of: unprimed MSCs (A); exposure of MSCs to a six component priming medium of the present disclosure (B); exposure of MSCs to a three component priming medium of the present disclosure (C); and exposure of MSCs to a 2× version of the three component priming medium (D). FIG. 7B shows the results of peritoneal lavage fluid NK cell percentage nine months after MSCs were primed with a priming medium of the present disclosure and MSC treatments showing dose effect for the priming medium and its sustainable effect on peritoneal NK cells;

FIGS. 8A-B show the results of MSC exposure to a priming medium of the present disclosure on different subsets of T cells in peritoneal lavage fluid in pristane induced SLE mouse model in vivo. FIG. 8A shows a remarkable increase in CD4+ FoxP3+ T regulatory cell percent (percents are multiplied by 10 for showing in graph) after MSC priming (OHA in FIG. 8A). FIG. 8B shows a sustainable immune system modulation (suppression of Th17 cell percent in peritoneal lavage) after nine months following a one-time injection with primed MSCs (1×10⁶ cells) over Th17 cells (CD4+ RORγ+ T cells);

FIG. 9 is a graph showing that plasmacytoid dendritic cell antigen-1+ cells (showing pDCs) are significantly reduced one week after administration of primed MSCs (Pristane-HXB-319 in FIG. 9) as compared to unprimed MSCs (Pristane-MSCs in graph) in peritoneal lavage fluid of pristane induced SLE mouse model in vivo. Furthermore, there is a significant dose effect when the cells were used in the amount of 2×10⁶ per mouse (Pristane-HXB-319 2X);

FIG. 10 is a graph showing that administration of primed MSCs has a positive effect on PD-L1+(CD-274) cells (anti-inflammatory and immune check point cell marker) in peritoneal lavage fluid percent count (multiplied by 10 to show on graph), showing evidence of an anti-inflammatory effect in pristane induced mice model;

FIG. 11 is a series of graphs showing that in an acute pristane mouse model, one-time injection of MSCs treated with a priming medium of the present disclosure (Pristane-HXB-319) (1×10⁶ cells) suppressed both systemic levels of IFN-α (left panel) and IFN-γ (right panel) in serum;

FIG. 12 is a series of graphs showing that in a chronic pristane mouse model (9 months after pristane induction), one-time injection of MSCs treated with priming medium of the present disclosure (Pristane-HXB-319, -2X or -2.5M) (1× or 2× or 2.5×10⁶ or cells) suppressed both systemic levels of IFN-α (top panel) and IFN-γ (bottom panel) in serum as compared to Pristane-MSC injections (1× or 2×10⁶ cells);

FIG. 13 is a series of graphs showing serum proinflammatory cytokine levels one week after injection with MSCs exposed to a priming medium of the present disclosure;

FIG. 14 is a graph showing IL-10 levels one week after injection with MSCs exposed to a priming medium of the present disclosure;

FIG. 15 is a graph showing serum levels of IL-17A after nine months of pristane injection and treatment with MSCs exposed to a priming medium of the present disclosure (indicated by OHA in FIG. 15);

FIG. 16 is a graph showing show double stranded DNA titer change. Treatment with MSCs exposed with a priming medium of the present disclosure shows significant control of double stranded DNA at higher doses nine months post-injection (after 2× or above 2.5 million cells per mouse, the dose effect was significant, OHA in graph represents MSCs primed with HXB-319);

FIG. 17 is a graph showing tapering B-cell activating factor (BAFF) levels in serum for MSCs exposed with a priming medium of the present disclosure;

FIG. 18 is a graph showing pathological evaluation scores for chronic glomerulonephritis in pristane-injected mice treated with a priming medium of the present disclosure;

FIG. 19 is a graph showing pathological evaluation scores for global and local scarring in pristane-injected mice treated with a priming medium of the present disclosure;

FIGS. 20A-B are a series of graphs showing IDO expression levels and activity following treatment of MSCs with variations of two- and three-component subsets of the priming media of the present disclosure;

FIGS. 21A-B are a series of graphs showing fold difference in gene expression (FIG. 21A) and IL-6/IL-8 levels (FIG. 21B) in primed and unprimed MSCs with variations of two- and three-component subsets of the priming media of the present disclosure;

FIG. 22 is a graph showing IL-10 and CD274 expression in MSCs treated with variations of two- and three-component subsets of the priming media of the present disclosure;

FIG. 23 is a graph showing TLR-7 and TLR-4 expression in MSCs treated with variations of two- and three-component subsets of the priming media of the present disclosure;

FIG. 24 is a series of graphs showing that RT-PCR results for IDO and CD274 expression in MSCs treated with HXB-319 and a three-component subset thereof are dose-dependent;

FIG. 25 is a graph showing differences in fractalkine suppression, AKT, and CTLA-4 between HXB-319 and a three-component subset thereof; and

FIG. 26 is a graph showing differences in IL-6 and IL-8 expression levels in MSCs primed with HXB-319 and a three-component subset thereof.

### DETAILED DESCRIPTION

The methods and techniques described herein are generally performed according to conventional methods well-known in the art and as described in various general and more specific references that are cited and discussed throughout the present specification unless otherwise indicated. See, e.g., Sambrook et al., Molecular Cloning: A Laboratory Manual, 3rd ed., Cold Spring Harbor Laboratory Press, Cold Spring Harbor, N.Y. (2001), Ausubel et al., Current Protocols in Molecular Biology, Greene Publishing Associates (1992), and Harlow and Lane, Antibodies: A Laboratory Manual, Cold Spring Harbor Laboratory Press, Cold Spring Harbor, N.Y. (1990).

### Definitions

For clarification in understanding and ease in reference a list of terms used throughout the brief description section and the remainder of the application have been compiled here. Some of the terms are well known throughout the field and are defined here for clarity, while some of the terms are unique to this application and therefore have to be defined for proper understanding of the application.

“A” or “an” means herein one or more than one; at least one. Where the plural form is used herein, it generally includes the singular.

As used herein, the term “cell bank” can refer to stem cells (e.g., mesenchymal stem cells, MSCs, or multipotent stromal cells) that have been cultured with a priming medium of the present disclosure and then stored for future use. Such cells may be stored in aliquots. They can be used directly out of storage or may be expanded after storage. This is a convenience so that there are “off the shelf” cells available for administration in research studies, clinical trials, or clinical therapies. These cell lines may already be stored in a pharmaceutically-acceptable excipient so they may be directly administered or they may be mixed with an appropriate excipient when they are released from storage. Cells may be frozen or otherwise stored in a form to preserve viability. Banks can be made using cells derived from the individual to be treated. Or banks can contain cells for allogeneic uses.

As used herein, the terms “cellular therapy” or “cell-based therapy” can refer to the transplantation of human or animal stem cells of the present disclosure (i.e., cells that have been cultured, contacted, or exposed to priming media of the present disclosure) to prevent, treat, or ameliorate one or more symptoms associated with a disease or disorder (e.g., a systemic inflammatory or autoinflammatory), including, but not limited to, the replacement or repair of damaged tissues or organs, the modulation of immune reactions and the reduction of inflammatory symptoms.

As used herein, the terms “repair” and “repairing” when used directly in reference to damaged tissues can refer to the amelioration of such damage by both direct mechanisms, such as the regeneration of damaged tissues, as well as through indirect mechanisms, e.g., reducing inflammation thereby enabling tissue formation by the stem cells of the present disclosure (i.e., cells that have been cultured, contacted, or exposed to priming media of the present disclosure).

As used herein, the terms “cell growth medium” can refer to an aqueous proliferation or expansion media containing the factors and nutrients suitable for supporting the growth of cells, such as stem cells of the present disclosure (i.e., cells that have been cultured, contacted, or exposed to priming media of the present disclosure) as described herein. For initial seeding and culture (e.g., to confluency), a standard, or off-the-shelf medium can be used, such as DMEM with 10% FBS. After confluency and for cell maintenance, a support medium, such as serum-free DMEM with low glucose, can be used. The cell culture media applied are well known in the art of cell cultures and all are commercially available.

The term “comprising” can mean, without other limitation, including the referent, necessarily, without any qualification or exclusion on what else may be included. For example, “a composition comprising x and y” encompasses any composition that contains x and y, no matter what other components may be present in the composition. Likewise, “a method comprising the step of x” encompasses any method in which x is carried out, whether x is the only step in the method or it is only one of the steps, no matter how many other steps there may be and no matter how simple or complex x is in comparison to them. “Comprised of and similar phrases using words of the root “comprise” are used herein as synonyms of “comprising” and have the same meaning, as does the word “includes.”

As used herein, the term “conditioned medium” or “CM” can refer to a medium that stem cells of the present disclosure (i.e., cells that have been cultured, contacted, or exposed to priming media of the present disclosure) have been cultured in for a period of time. In one example, conditioned medium can be prepared by culturing stem cells, generated by the priming methods disclosed herein, and then collecting the supernatant, or conditioned medium, after the culture period.

As used herein, the term “defined medium” can refer to a cell culture growth medium to which specific, indicated factors have been added. The term can also refer to a cell culture growth medium in which all components can be described by their chemical formulae and are present in known concentrations, in contrast to an undefined medium.

As used herein, the term “effective amount” can refer to an amount which provides the desired local or systemic effect, e.g., effective to ameliorate undesirable effects (e.g., inflammation associated with a systemic inflammatory or autoinflammatory disease, such as graft-versus-host disease, systemic lupus erythematosus, etc.), including achieving the specific desired effects described in the present disclosure. For example, an effective amount is an amount sufficient to effectuate a beneficial or desired clinical result. The effective amount can be provided all at once in a single administration or in fractional amounts that provide the effective amount in several administrations. The precise determination of what would be considered an effective amount may be based on factors individual to each subject, including their size, age, injury, and/or disease or injury being treated, and amount of time since the injury occurred or the disease began. One skilled in the art will be able to determine the effective amount for a given subject based on these considerations which are routine in the art. The term “effective dose” can mean the same as “effective amount.”

As used herein, the term “effective route” can refer to a route which provides for delivery of an agent (e.g., stem cells) to a desired compartment, system, or location in the body. For example, an effective route is one through which an agent can be administered to provide at the desired site of action an amount of the agent sufficient to effectuate a beneficial or desired clinical result.

As used herein, the term “exogenously added”, in the context of cultures or conditioned media, can refer to compounds, growth factors, differentiation factors, and the like, that are added to the cultures or media to supplement any compounds or growth factors that may already be present in the culture or media.

As used herein, the term “isolated” can refer to a cell or cells which is/are not associated with one or more cells or one or more cellular components that are associated with the cell or cells in vivo. An “enriched population” can mean a relative increase in numbers of a desired cell relative to one or more other cell types in vivo or in primary culture. However, as used herein, the term “isolated” does not indicate the presence of only the cells described herein. Rather, the term “isolated” can indicate that the cells described herein are removed from their natural tissue environment and are present at a higher concentration as compared to the normal tissue environment. Accordingly, an “isolated” cell population may further include cell types in addition to the cells described herein cells and may include additional tissue components. This also can be expressed in terms of cell doublings, for example. A cell may have undergone 10, 20, 30, 40 or more doublings in vitro or ex vivo to become enriched compared to its original numbers in vivo or in its original tissue environment (e.g., bone marrow, peripheral blood, placenta, umbilical cord, umbilical cord blood, adipose tissue, etc.).

As used herein, the term “mesenchymal stem cell” or “MSC” can refer to cells that are derived from the embryonal mesoderm and can be isolated from many sources, including adult bone marrow, peripheral blood, fat, placenta, and umbilical blood, among others. MSCs can differentiate into many mesodermal tissues, including muscle, bone, cartilage, fat, and tendon. There is considerable literature on these cells. See, for example, U.S. Pat. Nos. 5,486,389; 5,827,735; 5,811,094; 5,736,396; 5,837,539; 5,837,670; and 5,827,740. See also Pittenger, M. et al, Science, 284:143-147 (1999). In some instances, MSCs can be derived from an apparently healthy subject (e.g., a human subject); that is, a subject who has no signs and/or symptoms of a disease. The term can also be used interchangeably with “multipotent stromal cell”.

As used herein, the terms “multipotent” or “multipotent cells” refer to a cell type that can give rise to a limited number of other particular cell types. Multipotent cells are committed to one or more embryonic cell fates, and thus, in contrast to pluripotent cells, cannot give rise to each of the three embryonic cell lineages as well as extraembryonic cells.

When used in connection with cell cultures and/or cell populations, the term “portion” can mean any non-zero amount of the cell culture or cell population, which ranges from a single cell to the entirety of the cell culture or cells population. In some instances, the term “portion” can mean at least 5%, at least 6%, at least 7%, at least 8%, at least 9%, at least 10%, at least 11%, at least 12%, at least 13%, at least 14%, at least 15%, at least 16%, at least 17%, at least 18%, at least 19%, at least 20%, at least 21%, at least 22%, at least 23%, at least 24%, at least 25%, at least 26%, at least 27%, at least 28%, at least 29%, at least 30%, at least 31%, at least 32%, at least 33%, at least 34%, at least 35%, at least 36%, at least 37%, at least 38%, at least 39%, at least 40%, at least 41%, at least 42%, at least 43%, at least 44%, at least 45%, at least 46%, at least 47%, at least 48%, at least 49%, at least 50%, at least 51%, at least 52%, at least 53%, at least 54%, at least 55%, at least 56%, at least 57%, at least 58%, at least 59%, at least 60%, at least 61%, at least 62%, at least 63%, at least 64%, at least 65%, at least 66%, at least 67%, at least 68%, at least 69%, at least 70%, at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94% or at least 95% of the cell culture or cell population.

As used herein, the term “pharmaceutically acceptable” can refer to those compounds, materials, compositions (e.g., cells), and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

As used herein, the terms “pluripotent” or “multipotent”, in the context of a cell, can refer to a cell that gives rise to more than one tissue deriving from any of the three embryonic cell lineages as well as extraembryonic cells. The term is limited to the inner cell mass state, and is not used at any point to describe lineage restricted progenitors that form during development or are present from the perinatal to adult period. The pluripotent cell state is a natural one, but can be propagated in vitro under specific conditions known to those skilled in the art. In such culture conditions, perpetual maintenance of the stem cell, or pluripotent state, occurs. The defining qualities of the pluripotent cells are not limited to their functional characteristic, but can also be described through gene expression patterns and consequently the identification of pluripotent cells can be accomplished through staining patterns for markers they exhibit as well as quantitative assays. The presence of the protein transcription factors OCT3/4, SOX2 and NANOG represent a group of core transcription factors involved in maintaining the pluripotent state and the down regulation of these factors is a prerequisite for differentiation. These three transcription factors have all been shown to interact with one another and there is a great deal of overlap in the genes that are under their transcriptional control. In addition to the transcription factors responsible for pluripotent maintenance are often identified through the use of several surface markers present on them, the most common ones being Stage Specific Embryonic Antigens 3 and 4 (SSEA3 and SSEA4), Tra-1-61 and Tra-1-81. Pluripotent cells, such as embryonic stem cells, can also exhibit alkaline phosphatase activity, which is often used in the identification of pluripotent cells.

As used herein, the term “progenitor cell” can refer to a normal cellular state, at any point, which represented a direct lineage ancestor for a differentiated cell. By definition, progenitors are not irreversibly destined to adopt the said committed fate, but may be multipotent for more than one committed state. As such, they display competence for differentiation into more than a single descendant fate. As development proceeds in any organism, the gradual loss of competency, and thus loss of multipotency, is the hall mark of differentiation and the temporally defined formation of tissues and organs. One example of a progenitor cell can include an induced pluripotent stem cell (iPSC), which is a type of pluripotent stem cell artificially derived from a non-pluripotent cell, typically an adult somatic cell, by inserting certain genes.

As used herein, the term “self-renewal” can refer to the ability of a stem cell to produce replicate daughter stem cells having differentiation potential that is identical to those from which they arose.

As used herein, the terms “expand”, “expansion”, and “proliferation” can refer to the process that results in an increase of the number of cells. The term may also refer to the balance between cell divisions and cell loss through cell death or differentiation.

As used herein, the term “stem cell” can refer to a cell that can undergo self-renewal (i.e., progeny with the same differentiation potential) and also produce progeny cells that are more restricted in differentiation potential. Within the context of the present disclosure, a stem cell would also encompass a more differentiated cell that has de-differentiated, for example, by nuclear transfer, by fusion with a more primitive stem cell, by introduction of specific transcription factors, or by culture under specific conditions. See, for example, Wilmut et al., Nature, 385:810-813 (1997); Ying et al., Nature, 416:545-548 (2002); Guan et al., Nature, 440:1199-1203 (2006); Takahashi et al., Cell, 126:663-676 (2006); Okita et al., Nature, 448:313-317 (2007); and Takahashi et al., Cell, 131:861-872 (2007).

Dedifferentiation may also be caused by the administration of certain compounds or exposure to a physical environment in vitro or in vivo that would cause the dedifferentiation, and this process essentially describes a gain in multipotency. Stem cells also may be derived from abnormal tissue, such as a teratocarcinoma and some other sources such as embryoid bodies (although these can be considered embryonic stem cells in that they are derived from embryonic tissue, although not directly from the inner cell mass). The pluripotent stem cell state may also be created by introducing genes associated with stem cell function into a non-stem cell, such as an induced pluripotent stem cell.

As used herein, the term “subject” can refer to a vertebrate or a mammal. Examples of subjects can include livestock, test animals, and pets, such as ovine, bovine, porcine, canine, feline and murine mammals, as well as reptiles, birds and fish. The terms “patient” and “subject” can be used interchangeably herein. In one example, a subject is a mammal. The mammal can be a human, non-human primate, mouse, rat, dog, cat, horse, or cow, but are not limited to these examples.

As used herein, the term “substantially free of”, with respect to cells in cell cultures or in cell populations, can mean that the specified cell type of which the cell culture or cell population is free, is present in an amount of less than about 10%, less than about 9%, less than about 8%, less than about 7%, less than about 6%, less than about 5%, less than about 4%, less than about 3%, less than about 2% or less than about 1% of the total number of cells present in the cell culture or cell population.

As used herein, the term “therapeutically effective amount” can refer to the amount of an agent (e.g., a cell or cells that have been cultured, contacted, or exposed to priming media of the present disclosure) determined to produce any therapeutic response in a subject. Treatments that are therapeutically effective within the meaning of the term as used herein can include treatments that improve a subject's quality of life even if they do not improve the disease outcome per se. Such therapeutically effective amounts are readily ascertained by one of ordinary skill in the art. Thus, to “treat” can mean to deliver such an amount. Thus, treating can prevent or ameliorate any pathological symptoms of disease or condition, such as a systemic inflammatory or autoinflammatory disease.

As used herein, the terms “treat,” “treating,” or “treatment” can encompass, among others, preventing, ameliorating, inhibiting, or curing a deficiency, dysfunction, disease, or other deleterious process, including those that interfere with and/or result from a therapy. In some instances, the symptoms of a disease or disorder can be alleviated by at least 5%, at least 10%, at least 20%, at least 30%, at least 40%, or at least 50%.

As used herein, the terms “prevent”, “preventing”, “prophylactic”, or “prophylactically” can refer to completely or partially (e.g., by at least 5%, at least 10%, at least 20%, at least 30%, at least 40%, or at least 50%) preventing or inhibiting at least one symptom of a disease or disorder (e.g., a systemic inflammatory or autoinflammatory disease or disorder); or, the frequency with which such a symptom is exhibited can refer to completely or partially (e.g., by at least 5%, at least 10%, at least 20%, at least 30%, at least 40%, or at least 50%) preventing or inhibiting a symptom of the disease or disorder; or, the frequency with which such a symptom is exhibited.

As used herein, the term “induction”, when referring to a stem cell (or stem cells) contacted with a priming medium of the present disclosure, can refer to a change in the stem cell's (or stem cells') phenotype (e.g., surface marker type and/or presence and/or absence) and/or genotype (e.g., mRNA expression level) brought about as a result of contact with the priming medium.

As used herein, the term “homogenous induction” can refer to at least a portion of stem cells in a population of stem cells that are induced to obtain one or more changes in the stem cells' phenotype and/or genotype as a result of contact with a priming medium of the present disclosure. In some instances, substantially homogenous induction can occur. Substantially homogenous induction can refer to a majority of stem cells (>50%) that are induced to obtain one or more changes in the stem cells' phenotype and/or genotype. Alternatively, substantially homogenous induction can refer to a majority of stem cells, e.g., about 50-55%, about 55-60%, about 60-65%, about 65-70%, about 70-75%, about 75-80%, about 80-85%, about 85-90%, about 90-95%, about 96%, about 97%, about 98%, or about 99%, that are induced to obtain one or more changes in the stem cells' phenotype and/or genotype.

As used herein, the term “validate” can mean to confirm. In the context of the present disclosure, one can confirm that a stem cell is particular type cell (e.g., a MSC) with a desired potency and/or phenotype. This is so that one can then use that stem cell (in treatment, banking, drug screening, etc.) with a reasonable expectation of efficacy. Accordingly, to validate can mean to confirm that the stem cells, having been found to have/established as having the desired activity and/or phenotype, in fact, retain that activity and/or phenotype. Thus, validation can be a verification event in a two-event process involving the original determination and the follow-up determination. The second event can be referred to herein as “validation”.

As used herein, the term “off-the-shelf media” can refer to a media composition that includes a serum-containing basal medium with other compounds, e.g., ascorbic-acid, dexamethasone, etc. Serum, by nature, is an ill-defined component of a medium. Thus, the exact composition of commercially available “off-the-shelf”, serum-containing media is unknown due to the heterogeneity of the different lots of serum. One example of an “off-the-shelf media” is Dulbecco's modified Eagle's medium/Ham's Nutrient Mixture F-12 (DMEM/F-12 1:1; Invitrogen) supplemented with 1% L-glutamine (GlutaMAX; Invitrogen), 10% fetal bovine serum, 100 nM Dexamethsone, 50 μM Ascorbic acid-2P, and 10 mM β-Glycerophosphate.

As used herein, the terms “systemic autoimmune disease or disorder” or “autoinflammatory disease or disorder” can refer to a condition in a subject characterized by cellular, tissue and/or organ injury caused by an immunological reaction of the subject to its own cells, tissues, and/or organs. Illustrative, non-limiting examples of autoimmune diseases which can be treated with the immunomodulatory cells yielded by the present disclosure can include alopecia areata, ankylosing spondylitis, antiphospholipid syndrome, autoimmune Addison's disease, autoimmune diseases of the adrenal gland, autoimmune hemolytic anemia, autoimmune hepatitis, autoimmune oophoritis and orchitis, autoimmune thrombocytopenia, Behcet's disease, bullous pemphigoid, cardiomyopathy, celiac sprue-dermatitis, chronic fatigue immune dysfunction syndrome (CF1DS), chronic inflammatory demyelinating polyneuropathy, Churg-Strauss syndrome, cicatrical pemphigoid, CREST syndrome, cold agglutinin disease, discoid lupus, essential mixed cryoglobulinemia, fibromyalgia-fibromyositis, glomerulonephritis, Graves' disease, Guillain-Barre, Hashimoto's thyroiditis, idiopathic pulmonary fibrosis, idiopathic thrombocytopenia purpura (ITP), IgA neuropathy, juvenile arthritis, lichen planus, Meniere's disease, mixed connective tissue disease, multiple sclerosis, type 1 or immune-mediated diabetes mellitus, myasthenia gravis, pemphigus vulgaris, pernicious anemia, polyarteritis nodosa, polychondritis, polyglandular syndromes, polymyalgia rheumatica, polymyositis and dermatomyositis, primary agammaglobulinemia, primary biliary cirrhosis, psoriasis, psoriatic arthritis, Raynauld's phenomenon, Reiter's syndrome, sarcoidosis, scleroderma, progressive systemic sclerosis, Sjogren's syndrome, Good pasture's syndrome, stiff-man syndrome, systemic lupus erythematosus, lupus erythematosus, takayasu arteritis, temporal arteristis/giant cell arteritis, ulcerative colitis, uveitis, vasculitides such as dermatitis herpetiformis vasculitis, vitiligo, Wegener's granulomatosis, Anti-Glomerular Basement Membrane Disease, Antiphospholipid Syndrome, Autoimmune Diseases of the Nervous System, Familial Mediterranean Fever, Lambert-Eaton Myasthenic Syndrome, Sympathetic Ophthalmia, Polyendocrinopathies, Psoriasis, etc.

In one example, systemic autoimmune diseases or autoinflammatory diseases treatable by the present disclosure can include SLE, mixed connective tissue disease, Sjogren's syndrome, overlap syndrome (rheumatoid arthritis and SLE and/or Sjogren's syndrome), graft-versus-host disease, adult or juvenile onset psoriatic arthritis, dermatomyositis, polymyositis, other myositis, systemic vasculitis, CNS vasculitis, scleroderma, inflammatory bowel disease, Crohn's disease, ulcerative colitis, rheumatoid arthritis, systemic onset juvenile arthritis, juvenile arthritis, adult Stills disease and systemic sclerosis.

Other examples of systemic autoimmune diseases or autoinflammatory diseases can include monogenic interferonopathies, where gene variants cause activation of Type I interferon pathway and thereby result in complex rheumatic diseases. These diseases are considered to lie on an autoimmune-autoinflammation spectrum that depends on the driver of dysregulated Type I interferon production. These include Aicardi-Goutieres syndrome (AGS), chronic atypical neutrophilic dermatosis with lipodystrophy and elevated temperature (CANDLE), and STING-associated vasculopathy with onset in infancy (SAVI). Others diseases associated with vasculitis and high interferon levels include retinal vasculopathy with cerebral leukodystrophy (RVCL) and Singleton-Merten syndrome, where the clinical manifestations are thought to relate to chronic inflammation, at least in part conferred by constitutive activation of RIG-I resulting in increased Type I interferon activity and ISG expression.

As used herein, the term “immunomodulatory” can refer to the modification, amplification, inhibition or reduction of one or more biological activities of the immune system which includes, but is not limited to, downregulation of immune response, augmentation of immune responses and changes of the inflammatory states mediated by changes in cytokine profile, cytotoxic activity and antibody production and their effects on immune and immune related cells.

As used herein, the term “population of cells” can refer to any number of stem cells (e.g., MSCs) greater than 1, but is preferably at least 1×10³ cells, at least 1×10⁴ cells, at least 1×10⁵ cells, at least 1×10⁶ cells, at least 1×10⁷ cells, at least 1×10⁸ cells, or at least 1×10⁹ cells. In some instances, at least 50%, at least 55%, at least 60%, at least 65%, at least 70%, at least 75%, at least 80%, at least 85%, at least 90%, at least 91%, at least 92%, at least 93%, at least 94% or at least 95%, of a population of cells (% by cell number) (e.g., stem cell, such as MSCs) in an initial population of stem cells will be undifferentiated stem cells (e.g., undifferentiated MSCs).

As used herein, the term “significant expression” or its equivalent terms “positive” and “+” when used regarding a cell surface marker means that, in a cell population, more than 20%, preferably more than 30%, 40%, 50%, 60%, 70%, 80%, 90% 95%, 98%, 99%, or even 100% of the cells express the cell surface marker.

Expression of cell surface markers may be determined, for example, by means of flow cytometry for a specific cell surface marker using conventional methods and apparatus (for example a BECKMAN COULTER EPICS XL FACS system used with commercially available antibodies and standard protocols known in the art) that show a signal for a specific cell surface marker in flow cytometry above the background signal using conventional methods and apparatus. The background signal is defined as the signal intensity given by a non-specific antibody of the same isotype as the specific antibody used to detect each surface marker in conventional FACS analysis. For a marker to be considered positive the specific signal observed is stronger than 20%, preferably stronger than, 30%, 40%, 50%, 60%, 70%, 80%, 90%, 500%, 1000%, 5000%, 10000% or above, than the background signal intensity using conventional methods and apparatus. Furthermore, commercially available and known monoclonal antibodies against said cell-surface markers (e.g., cellular receptors and transmembrane proteins) can be used to identify relevant cells.

As used herein, the term “priming medium” can refer to a cell culture medium that contains novel and specific components whose combination synergistically induces an anti-inflammatory phenotype in stem cells (e.g., MSCs) contacted therewith under effective culture conditions.

As used herein, the term “Type I interferon pathway” can refer to an inflammatory pathway responsible for control of innate and adaptive immunity. Type I interferons (IFNs) (mainly IFN-α and IFN-β) regulate the Type I interferon pathway. Type I IFN production is induced after detection of pattern-recognition receptors (PRRs). Type I interferons modulate innate immune responses in a balanced manner that promote antigen presentation and natural killer cell functions while restraining pro-inflammatory pathways and cytokine production. Type I interferons also activate the adaptive immune system, thus promoting the development of high-affinity antigen-specific T and B cell responses and immunological memory and start a cascade of events, which can result in autoimmune diseases (e.g., Sjogren's syndrome or SLE).

As used herein, the term “Type II interferon pathway” can refer to an inflammatory pathway that is primarly regulated by interferon gamma (IFN-γ). IFN-γ primarily signals through the Jak-Stat pathway, a pathway that affects gene regulation. Jak-Stat signaling involves sequential receptor recruitment and activation of members of the Janus family of kinases (Jaks: Jaks 1-3 and Tyk2) and the Stats (Stats 1-6, including Stat5a and Stat5b) to control transcription of target genes via specific response elements. IFN-γ is a cytokine that has an important role in adaptive and innate immunity.

As used herein, the term “functional activator of a Type I and Type II interferon pathway” can refer to any one or a combination of molecules that stimulates both the Type I and Type II interferon pathways. In one example, the expression level and/or activity of IDO can be used as a measure of Type I and Type II interferon pathway activation (e.g., increased IDO expression and/or activity, as compared to a control value, can indicate activation of the Type I and Type II interferon pathways). Methods for assaying IDO expression and activity are known in the art.

As used herein, the term “anti-inflammatory phenotype” can refer to the conglomerate of multiple cellular processes involving gene and protein expression that result in a cell's particular morphological and functional characteristics which, as a result of culture with a priming medium of the present disclosure, produce a cell marked or characterized by increased expression and/or secretion of one or more anti-inflammatory or immune modulatory mediators or markers as compared to a cell that has not been contacted with a priming medium of the present disclosure.

As used herein, the term “pro-inflammatory cytokine” can refer to a type of signaling molecule that is secreted from immune cells (e.g., helper or cytotoxic T cells (Th), monocytes, dendritic cells and macrophages) and which stimulates inflammation.

As used herein, the term “interferon-regulated autoimmune disease” can refer to an autoimmune disease where one or a combination of interferons actively play a role in the etiology or pathogenesis of the disease to the point of autoimmune disease development. In other words, if interferon activity is the hallmark of the disease, then the disease is an interferon-regulated autoimmune disease.

As used herein, the term “abnormal activation of a Type I interferon pathway” can refer to a response by the innate and adaptive immune systems that is different than what is typically expected during activation of the Type I interferon pathway. For example, an exaggerated innate and adaptive immune response can occur as a result of abnormal activation of the Type I interferon pathway, such as a cytokine storm.

As used herein, the term “abnormal activation of a Type II interferon pathway” can refer to refer to a response by the innate and adaptive immune systems that is different than what is typically expected during activation of the Type I interferon pathway. For example, an exaggerated or deleterious innate and adaptive immune response can occur in the absence of pathogen induction, thereby leading to end organ inflammatory changes and damage.

As used herein, the term “CAR-T cells” can refer to T cells that have been taken from a subject, been modified in vitro to have a chimeric antigen receptor (CAR) protein added to the subject's T cells, and then administered back to the subject. Methods for priming CAR-T cells prior to administration back to the subject are disclosed herein.

As used herein, the term “CAR-NK cells” can refer to NK cells that have been taken from a subject, been modified in vitro to have a CAR protein added to the subject's NK cells, and then administered back to the subject. Methods for priming CAR-NK cells prior to administration back to the subject are disclosed herein.

As used herein, the term “immune modulatory mediator” can refer to a molecule (e.g., a cell-secreted molecule) that has an effect on one or more components of the immune system. Examples of immune system components can include proteins (e.g., cytokines, interleukins, antibodies, complement, cell receptors, ligands, immunohistocompatability complexes, etc.) as well as immune cells (e.g., lymphocytes, such as NK cells, T cells and B cells, neutrophils and macrophages/monocytes). In some instances, the effect can be positive (e.g., an increase in protein level and/or activity or an increase in immune cell proliferation and/or activity) or negative (e.g., a decrease in protein level and/or activity or an increase in immune cell proliferation and/or activity). Non-limiting examples of immune modulatory mediators can include IDO, IL-1β, IL-17A, TNF-α and TNF-γ.

As used herein, the term “cytotoxic activity” can refer to the ability of a cell (e.g., a CAR-T cell or a CAR-NK cell) to kill another cell of a different type, typically by means of elaborating porins in the case of a lymphocyte. This capability can be measured by co-culturing lymphocytes and target cells. Other assay methods for determining cytotoxic activity of a cell are known in the art.

As used herein, the terms “downregulating” or “reducing” an activity and/or amount of a molecule (e.g., an immune modulatory mediator) can refer to a downregulation, decrease, or reduction of at least 10%, at least 20%, at least 30%, at least 40%, at least 50% at least 60%, at least 70%, at least 80% or even at least 90% of the activity and/or amount of the molecule as compared to a control value. In some instances, the term “downregulating” can also refer to full inhibition.

As used herein, the terms “upregulating” or “increasing” an activity and/or amount of a molecule (e.g., an immune modulatory mediator) can refer to an upregulation or increase of at least 10%, at least 20%, at least 30%, at least 40%, at least 50% at least 60%, at least 70%, at least 80% or even at least 90% of the activity and/or amount of the molecule as compared to a control value.

Priming Media

One aspect of the present disclosure can include a priming medium for creating, from an unprimed population of stem cells, an isolated population of stem cells having an anti-inflammatory phenotype as compared to the population of unprimed stem cells. The priming medium can comprise a serum-free medium, a functional activator of a Type I IFN pathway and a Type II IFN pathway, and at least two pro-inflammatory cytokines. The functional activator and the at least two pro-inflammatory cytokines can be present in an amount sufficient to promote induction of the stem cells having an anti-inflammatory phenotype. The cells having an anti-inflammatory phenotype can be marked by increased expression and/or secretion of one or more anti-inflammatory or immune modulatory mediators as compared to the unprimed population of stem cells.

In some instances, a priming medium of the present disclosure can be a defined medium.

The priming medium can be a serum-free medium. Examples of a serum-free medium that can be included in a priming medium of the present disclosure can include DMEM-F12, DMEM, and alpha-MEM. A serum-free medium is one that contains no animal serum of any type. Serum-free media may be preferred to avoid possible xeno-contamination of the stem cells. A serum replacement-free medium is one that has not been supplemented with any commercial serum replacement formulation. The serum-free medium can be provided in the priming medium so that the percentage (e.g., w/v) of the serum-free medium comprising the priming medium is at least about 80-99%, for example, about 80-82%, about 82-84%, about 84-86%, about 86-88%, about 88-90%, about 90-92%, about 92-94%, about 94-96%, about 96-98%, or about 98-99%.

Priming media of the present disclosure can include other components needed for cell survival and contamination prevention, such as glucose and antimicrobial agents used to mitigate bacterial, mycoplasmal, and fungal contamination. Thus, priming media of the present disclosure can contain one or more antimicrobial agents or antibiotics to prevent contamination. Typically, antibiotics or anti-mycotic compounds used are mixtures of penicillin/streptomycin, but can also include, but are not limited to amphotericin (Fungizone®), ampicilhn, gentamicin, bleomycin, hygromacin, kanamycin, mitomycin, etc.

In some instances, a priming medium of the present disclosure can include one or more functional activators of a Type I IFN pathway and a Type II IFN pathway. In one example, a priming medium of the present disclosure includes only one functional activator of a Type I IFN pathway and a Type II IFN pathway (e.g., Poly (I:C)). In some instances, a functional activator of a Type I IFN pathway and a Type II IFN pathway can include a toll-like receptor (TLR) ligand, such as TLR3. A TLR ligand can include any molecule, compound, or agent capable of activating TLR signaling and inducing an inflammatory response. A TLR ligand can activate TLR signaling by binding to, or interacting with, any of the known TLRs, such as TLR1-TLR10). a functional activator of a Type I IFN pathway and a Type II IFN pathway can include a synthetic molecule (e.g., a synthetic analog of a TLR ligand) that stimulates or activates a Type I IFN pathway and a Type II IFN pathway. In some instances, a TLR ligand can include Poly (I:C), lipopolysaccharide (LPS), tumor necrosis factor-alpha (TNF-α), and interleukin-1β (IL-1β). In one example, a TLR3 receptor can be induced by Poly (I:C). In another example, a TLR4 receptor can be induced by LPS.

In one example, a priming medium of the present disclosure can include Poly (I:C) as the functional activator of a Type I IFN pathway and a Type II IFN pathway. As described in Example 1 below, it was surprisingly discovered that Poly (I:C) exposure of human MSC cultures resulted in: increasing IL-1β production (which induced CCR-8 activation); five canonical pathways that were activated and three that were downregulated; and significant expansion of CD146+/pericyte-like MSCs.

One or more functional activators of a Type I IFN pathway and a Type II IFN pathway can be included in a priming medium of the present disclosure at a concentration sufficient to activate the Type I and Type II IFN pathways. For example, a functional activator of a Type I IFN pathway and a Type II IFN pathway can be included in the priming medium at a concentration of at least about 0.1 μg/ml to about 500 μg/ml, about 0.2 μg/ml to about 450 μg/ml, about 0.3 μg/ml to about 400 μg/ml, about 0.4 μg/ml to about 350 μg/ml, about 0.5 μg/ml to about 300 μg/ml, about 0.6 μg/ml to about 250 μg/ml, about 0.7 μg/ml to about 200 μg/ml, about 0.8 μg/ml to about 150 μg/ml, about 0.9 μg/ml to about 100 μg/ml, about 0.5 μg/ml to about 50 μg/ml, about 0.5 μg/ml to about 25 μg/ml, about 0.5 μg/ml to about 10 μg/ml, or about 0.5 μg/ml to about 5 μg/ml. In one example, Poly (I:C) can be provided in a priming media of the present disclosure at a concentration of 1 μg/ml.

In some instances, a priming medium of the present disclosure can include at least two proinflammatory cytokines, at least three proinflammatory cytokines, at least four proinflammatory cytokines, at least five proinflammatory cytokines, at least six proinflammatory cytokines, at least seven proinflammatory cytokines, at least eight proinflammatory cytokines, or more than eight pro-inflammatory cytokines. Non-limiting examples of pro-inflammatory cytokines can include IFN-α, -β, -γ, TNF-α, -β, IL-1β, IL-1Ra, GM-CSF, IL-2, IL-3, IL-4, IL-6, IL-8, IL-10, IL-13, IL-15, IL-22, -23, MIP-1α/β, MCP-1, IP-10, TGF-β1, SCM-1 and IL-17A.

At least two proinflammatory cytokines can be included in a priming medium of the present disclosure at a concentration sufficient to promote induction of stem cells (e.g., MSCs) having an anti-inflammatory phenotype as compared to stem cells that have not been contacted with a priming medium of the present disclosure. For example, at least two proinflammatory cytokines can be included in the priming medium at a concentration of at least about 1 ng/ml to about 200 ng/ml, for example, about 1-10 ng/ml, about 10-20 ng/ml, about 20-30 ng/ml, about 30-40 ng/ml, about 40-50 ng/ml, about 50-60 ng/ml, about 60-70 ng/ml, about 70-80 ng/ml, about 80-90 ng/ml, about 90-100 ng/ml, about 100-125 ng/ml, about 125-150 ng/ml, about 150-175 ng/ml, or about 175-200 ng/ml. In one example, a priming medium of the present disclosure can include IFN-γ (100 ng/ml) and TNF-α (20 ng/ml). In another example, a priming medium of the present disclosure can include IFN-γ (100 ng/ml), TNF-α (20 ng/ml), IL-1β (10 ng/ml) and IL-17A (50 ng/ml).

In some instances, a priming media of the present disclosure can only include serum-free medium, a functional activator of a Type I and Type II IFN pathway, at least two pro-inflammatory cytokines (e.g., at least four pro-inflammatory cytokines) and/or an essential vitamin. By “only”, it is meant that the priming medium does not include any other immune modulatory mediators (e.g., growth factors and cytokines) that would materially affect the efficacy (e.g., the basic and novel properties) of the priming medium, as described herein (e.g., in Examples 1 and 2); however, it will be understood that use of the term “only” can still permit inclusion of some components that do not materially affect the basic and novel properties of the priming media of the present application, such as those needed for cell survival during culture (e.g., amino acids, trace metals, inorganic salts, a carbon energy source, buffer, etc.). In such instances, it can be said that a priming medium of the present application consists essentially of a serum-free medium, a functional activator of a Type I and Type II IFN pathway, at least two pro-inflammatory cytokines (e.g., at least four pro-inflammatory cytokines) and/or an essential vitamin.

In one example of the present disclosure, a priming medium can include a serum-free medium, a functional activator of a Type I and Type II IFN pathway (e.g., Poly (I:C)), and two pro-inflammatory cytokines (e.g., IFN-γ and TNF-α). Such a priming medium is referred to herein as a “four-component priming medium”. In some instances, the four-component priming medium can only include a serum-free medium, a functional activator of a Type I and Type II IFN pathway (e.g., Poly (I:C)), and two pro-inflammatory cytokines (e.g., IFN-γ and TNF-α).

In another aspect, a priming medium of the present disclosure can comprise a serum-free medium, a functional activator of a Type I IFN pathway and a Type II IFN pathway, at least four pro-inflammatory cytokines, and an essential vitamin. The functional activator and the at least four pro-inflammatory cytokines can be present in an amount sufficient to promote induction of stem cells having an anti-inflammatory phenotype. The cells having an anti-inflammatory phenotype can be marked by increased expression and/or secretion of one or more anti-inflammatory or immune modulatory mediators as compared to the unprimed population of stem cells.

In some instances, a priming medium of the present disclosure can include at least one essential vitamin, such as ascorbic acid, thiamine, folic acid and riboflavin, as well as others known in the art. At least one essential vitamin can be present in the priming medium at a concentration of about 50-300 μM, about 50-75 μM, about 75-100 μM, about 100-125 μM, about 125-150 μM, about 150-175 μM, about 175-200 μM, about 200-225 μM, about 225-250 μM, about 250-275 μM, or about 275-300 μM. In one example, a priming medium of the present disclosure can include ascorbic acid (200 μM).

In one example of the present disclosure, a priming medium can include a serum-free medium, a functional activator of a Type I and Type II IFN pathway (e.g., Poly (I:C)), four pro-inflammatory cytokines (e.g., IFN-γ, TNF-α, IL-1β and IL-17A), an an essential vitamin (e.g., ascorbic acid). Such a priming medium is referred to herein as a “six-component priming medium”. In some instances, the six-component priming medium can only include a serum-free medium, a functional activator of a Type I and Type II IFN pathway (e.g., Poly (I:C)), four pro-inflammatory cytokines (e.g., IFN-γ, TNF-α, IL-1β and IL-17A), and an essential vitamin (e.g., ascorbic acid).

In some instances, priming media of the present disclosure can be formulated in deionized, distilled water. Priming media of the present disclosure can be sterilized prior to use to prevent contamination, e.g., by ultraviolet light, heating, irradiation or filtration. Priming media of the present disclosure may be frozen (e.g., at −20° C. or −80° C.) for storage or transport.

Priming media of the present disclosure can be a 1× formulation or a concentrated formulation, e.g., a 2× to 250× concentrated medium formulation. In a 1× formulation, each component in the priming medium is at the concentration intended for cell induction. In a concentrated formulation, one or more of the components is present at a higher concentration than intended for cell induction. Priming media of the present disclosure can be concentrated using known methods, e.g., salt precipitation or selective filtration. A concentrated priming medium may be diluted for use with water (preferably deionized and distilled) or any appropriate solution, e.g., an aqueous saline solution, an aqueous buffer or a culture medium.

Another aspect of the present disclosure can include a composition comprising a priming medium and stem cells (e.g., MSCs). In some instances, the priming medium can comprise a serum-free medium, a functional activator of a Type I IFN pathway and a Type II IFN pathway, and at least two pro-inflammatory cytokines. In other instances, the priming medium can comprise a serum-free medium, a functional activator of a Type I IFN pathway and a Type II IFN pathway, at least four pro-inflammatory cytokines, and an essential vitamin. The functional activator and the pro-inflammatory cytokines can be present in an amount sufficient to promote induction of stem cells having an anti-inflammatory phenotype. The cells having an anti-inflammatory phenotype can be marked by increased expression and/or secretion of one or more anti-inflammatory or immune modulatory mediators as compared to the unprimed population of stem cells.

In one example, a composition can comprise a four-component priming medium (e.g., only a four-component priming medium) and stem cells (e.g., MSCs).

In another example, a composition can comprise a six-component priming medium (e.g., only a six-component priming medium) and stem cells (e.g., MSCs).

Culture Methods

Another aspect of the present disclosure can include an in vitro method for creating, from a population of unprimed stem cells, an isolated population of stem cells having an anti-inflammatory phenotype as compared to the unprimed population of stem cells. The method can include contacting an unprimed population of stem cells with a priming medium for a time and under conditions sufficient to promote induction (e.g., homogenous or substantially homogenous induction) of stem cells having an anti-inflammatory phenotype. In some instances, the priming medium can comprise a serum-free medium, a functional activator of a Type I IFN pathway and a Type II IFN pathway, and at least two pro-inflammatory cytokines. In other instances, the priming medium can comprise a serum-free medium, a functional activator of a Type I IFN pathway and a Type II IFN pathway, at least four pro-inflammatory cytokines, and an essential vitamin. The created cells can have an anti-inflammatory phenotype and be marked by increased expression of one or more anti-inflammatory or immune modulatory mediators as compared to unprimed stem cells.

In one example of the in vitro method, the priming medium can comprise a four-component priming medium (e.g., only a four-component priming medium).

In another example of the in vitro method, the priming medium can comprise a six-component priming medium (e.g., only a six-component priming medium).

In some instances, the unprimed population of stem cells can include an isolated population of stem cells that have not been contacted with, exposed to, or cultured with a priming medium of the present disclosure. Stem cells used in the method can be autologous, allogeneic or xenogeneic. Alternatively, stem cells can be obtained from a commercial source (e.g., an established cell line). Other sources of stem cells are known to those skilled in the art. In one example, the unprimed stem cells include an isolated population of MSCs or multipotent stromal cells derived from tissue (e.g., adipose tissue, bone marrow or umbilical cord blood) of an apparently healthy subject (e.g., a human).

Unprimed stem cells can be cultured with priming media of the present disclosure using general cell culture methodologies can be appreciated by those skilled in the art. For example, unprimed stem cells can be isolated from a subject, or obtained from a commercial source, and then seeded in a culture vessel at a desired density and under appropriate ambient conditions (e.g., 37° C. in a humidified atmosphere and 5% CO₂). Cell culture can be performed using any suitable cell culture vessel as a support. Cell culture vessels of various shapes and sizes (e.g., flasks, single or multiwell plates, single or multiwell dishes, bottles, jars, vials, bags, bioreactors) and constructed from various different materials (e.g., plastic, glass) are known in the art. A suitable cell culture vessel can readily be selected by the skilled artisan.

Unprimed stem cells can be contacted with, exposed to, or cultured with a priming medium of the present disclosure for a desired period of time sufficient to promote induction of stem cells having an anti-inflammatory phenotype. The desired period of time can be one day or less (e.g., 6 or 12 hours), about 2 days (e.g., 18 hours), 2 days, 3 days, 4 days, or more than 5 days.

In some instances, it may be desirable to assay the unprimed stem cells, prior to contact with the priming medium, to verify the phenotype of the unprimed stem cells. For example, where the unprimed stem cells comprise MSCs, one or more known assays can be performed to verify that the presence of MSCs. In one example, flow cytometric evaluation of the presence of cell surface markers CD90, CD73, CD105 and absence of CD34, HLA-DR can be used to validate the presence of MSCs.

Methods for verifying the identity of cell populations—whether unprimed stem cells or primed stem cells—are known in the art. For example, the expression of certain markers can be determined by measuring the level at which the marker is present in the cells of a cell culture or a cell population. In such processes, the measurement of marker expression can be qualitative or quantitative. One method of quantitating the expression of markers that are produced by marker genes is through the use of quantitative PCR (Q-PCR). Methods of performing Q-PCR are well known in the art. Other methods which are known in the art can also be used to quantitate marker gene expression. For example, the expression of a marker gene product can be detected by using antibodies specific for the marker gene product of interest. Qualitative or semi-quantitative techniques, such as blot transfer methods and immunocytochemistry, can be used to measure marker expression. Additionally, it will be appreciated that techniques for measuring extracellular marker content, such as ELISA, may be utilized.

In another aspect, primed stem cells produced by the methods of the present disclosure can be enriched, isolated and/or purified according to techniques known to those of skill in the art. In some instances, cell populations enriched, isolated and/or purified for primed stem cells can be produced by isolating such cells from cell cultures. Primed stem cells produced by the methods described herein can be enriched, isolated and/or purified by using an affinity tag that is specific for such cells. Examples of affinity tags specific for primed stem cells of the present disclosure include antibodies, antibody fragments, ligands or other binding agents that are specific to a marker molecule, such as a polypeptide, that is present on the cell surface of the primed stem cells (e.g., CD146) but which is not present (or substantially present) on other cell types that would be found in a cell culture produced by the methods described herein.

Methods for making antibodies and using them for cell isolation are known in the art and such methods can be implemented for use with the antibodies and primed stem cells described herein. In one process, an antibody that binds to marker expressed by the primed stem cells can be attached to a magnetic bead and then allowed to bind to primed stem cells in a cell culture, which has been enzymatically treated to reduce intercellular and substrate adhesion. The cell/antibody/bead complexes are then exposed to a movable magnetic field, which is used to separate bead-bound primed stem cells from unbound cells. Once the primed stem cells are physically separated from other cells in culture, the antibody binding is disrupted and the cells are replated in appropriate tissue culture medium. If desired, the isolated cell compositions can be further purified by using an alternate affinity-based method or by additional rounds of enrichment using the same or different markers that are specific for the primed stem cells.

Any of the steps and procedures for isolating primed stem cells created by the methods of the present disclosure can be performed manually, if desired. Alternatively, the process of isolating such cells can be facilitated and/or automated through one or more suitable devices, examples of which are known in the art.

Using the methods described herein, cell populations or cell cultures can be enriched in primed stem cell content by at least about 2- to about 1000-fold as compared to unprimed or un-enriched cell populations or cell cultures. In some instances, primed stem cells can be enriched by at least about 5- to about 500-fold as compared to unprimed or un-enriched cell populations or cell cultures. In other instances, primed stem cells can be enriched from at least about 10- to about 200-fold as compared to unprimed or un-enriched cell populations or cell cultures. In still other instances, primed stem cells can be enriched from at least about 20- to about 100-fold as compared to unprimed or un-enriched cell populations or cell cultures. In yet other instances, primed cells can be enriched from at least about 40- to about 80-fold as compared to unprimed or un-enriched cell populations or cell cultures. In certain instances, primed stem cells can be enriched from at least about 2- to about 20-fold as compared to unprimed or un-enriched cell populations or cell cultures.

Additional aspects relate to compositions, such as cell cultures or cell populations, produced by the methods described herein and which comprise primed stem cells as the majority cell type. In some instances, the methods described herein produce cell cultures and/or cell populations comprising at least about 99%, at least about 98%, at least about 97%, at least about 96%, at least about 95%, at least about 94%, at least about 93%, at least about 92%, at least about 91%, at least about 90%, at least about 89%, at least about 88%, at least about 87%, at least about 86%, at least about 85%, at least about 84%, at least about 83%, at least about 82%, at least about 81%, at least about 80%, at least about 79%, at least about 78%, at least about 77%, at least about 76%, at least about 75%, at least about 74%, at least about 73%, at least about 72%, at least about 71%, at least about 70%, at least about 69%, at least about 68%, at least about 67%, at least about 66%, at least about 65%, at least about 64%, at least about 63%, at least about 62%, at least about 61%, at least about 60%, at least about 59%, at least about 58%, at least about 57%, at least about 56%, at least about 55%, at least about 54%, at least about 53%, at least about 52%, at least about 51% or at least about 50% primed stem cells. In other instances, the methods described herein produce cell cultures or cell populations comprising at least about 50%, at least about 45%, at least about 40%, at least about 35%, at least about 30%, at least about 25%, at least about 24%, at least about 23%, at least about 22%, at least about 21%, at least about 20%, at least about 19%, at least about 18%, at least about 17%, at least about 16%, at least about 15%, at least about 14%, at least about 13%, at least about 12%, at least about 11%, at least about 10%, at least about 9%, at least about 8%, at least about 7%, at least about 6%, at least about 5%, at least about 4%, at least about 3%, at least about 2% or at least about 1% primed stem cells. In some instances, the percentage of primed stem cells in the cell cultures or populations is calculated without regard to cells remaining in the culture.

In some instances, in vitro methods of the present disclosure can be performed in the presence or abscence of a layer of feeder cells (e.g., a layer of cells that are typically unable to divide and which provide extracellular secretions to help another cell to proliferate), such as fibroblast cells.

It will be appreciated that the steps of the methods disclosed herein can be performed in any suitable order or at the same time, as appropriate, and need not be performed in the order in which they are listed. For example, in the above method, the step of providing a population of unprimed stem cells may be performed before, after, or at the same time as, the step of providing a priming medium.

Primed Cells

Another aspect of the present disclosure includes an isolated population of stem cells having an anti-inflammatory phenotype as a result of contact with, exposure to, or culture with a priming medium of the present disclosure. Such cells, referred to as primed stem cells (e.g., primed MSCs), are marked by increased expression and/or secretion of one or more anti-inflammatory mediators and/or unique surface markers as compared to an unprimed population of stem cells. As discussed above, primed stem cells of the present disclosure are markedly different than naturally-occurring stem cells, at least because the primed stem cells have been contacted with, exposure to, or cultured with a priming medium having a unique combination components in serum-free media (and, in some instances, as a defined media), which is unlike an in vivo inflammatory state where numerous undefined molecules (e.g., proteins, carbohydrates, metabolites, etc.) are present. Consequently, primed stem cells of the present disclosure surprisingly possess unique functional and structural features and effects (identified below) that markedly distinguish them from naturally-occurring stem cells.

In some instances, the present disclosure can include an isolated population of primed MSCs (e.g., using a four- or six-component priming media) that are CD146+. In other instances, the present disclosure can include an isolated population of primed MSCs (e.g., using a four- or six-component priming media) that are CD146+ and Lep-R+. In further instances, the present disclosure can include an isolated population of primed MSCs (e.g., using a four- or six-component priming media) that are CD146+, Lep-R+, Nestin+ and Sdf-1+. In further instances, the present disclosure can include an isolated population of primed MSCs (e.g., using a four- or six-component priming media) that are CD146+ and at least one or a combination of Lep-R+, Nestin+ and Sdf-1+.

In some instances, present disclosure can include an isolated population of primed stem cells (e.g., MSCs) (e.g., using a six-component priming media) have an anti-inflammatory phenotype marked by increased expression of IDO, CD274, CD146 and platelet-derived growth factor receptor beta (PDGFRB), and by decreased expression of C-X3-C motif chemokine ligand 1 (CX3CL1), as compared to unprimed stem cells (e.g., unprimed MSCs). In one example, primed MSCs (e.g., using a six-component priming media) exhibit IDO expression that is about 50,000 to about 150,000 times greater than IDO expression as compared to unprimed MSCs.

In some aspects, an isolated population of primed stem cells (e.g., MSCs) (e.g., created using a four- or six-component priming media) can upregulate or downregulate production of immune cells and/or immune modulatory mediators.

In some instances, present disclosure can include an isolated population of primed stem cells (e.g., MSCs) (e.g., using a six-component priming media) that upregulate production of cytotoxic T cells by at least 4-fold, at least 5-fold, at least 6-fold, at least 7-fold, or at least 8-fold as compared to unprimed stem cells. In one example, primed MSCs (e.g., using a six-component priming media) upregulate production of cytotoxic T cells by at least 6-fold as compared to unprimed MSCs.

In some instances, present disclosure can include an isolated population of primed stem cells (e.g., MSCs) (e.g., using a six-component priming media) that upregulate production of Th1 cells by at least 4-fold, at least 5-fold, at least 6-fold, at least 7-fold, at least 8-fold, at least 9-fold, at least 10-fold, at least 11-fold, at least 12-fold, at least 13-fold, at least 14-fold, or at least 15-fold as compared to unprimed stem cells. In one example, primed MSCs (e.g., using a six-component priming media) upregulate production of cytotoxic T cells by at least 12-fold as compared to at least unprimed MSCs.

In some instances, present disclosure can include an isolated population of primed stem cells (e.g., MSCs) (e.g., using a six-component priming media) that upregulate production of CD4+ T cells by at least 4-fold, at least 5-fold, at least 6-fold, at least 7-fold, at least 8-fold, at least 9-fold, at least 10-fold, at least 11-fold, at least 12-fold, at least 13-fold, at least 14-fold, or at least 15-fold as compared to unprimed stem cells. In one example, primed MSCs (e.g., using a six-component priming media) upregulate production of CD4+ T cells by at least 12-fold as compared to at least unprimed MSCs.

In some instances, present disclosure can include an isolated population of primed stem cells (e.g., MSCs) (e.g., using a six-component priming media) that upregulate production of NK cells greater than unprimed stem cells.

In some instances, present disclosure can include an isolated population of primed stem cells (e.g., MSCs) (e.g., using a six-component priming media) that suppress production of Th17 cells greater than unprimed stem cells.

In some instances, present disclosure can include an isolated population of primed stem cells (e.g., MSCs) (e.g., using a six-component priming media) that suppress production of double-stranded DNA autoantibodies in vivo as compared to unprimed stem cells.

In some instances, present disclosure can include an isolated population of primed stem cells (e.g., MSCs) (e.g., using a six-component priming media) that suppress production of B-cell activating factor (BAFF) as compared to unprimed stem cells.

In some instances, present disclosure can include an isolated population of primed stem cells (e.g., MSCs) (e.g., using a six-component priming media) that decrease production of IL-17 as compared to unprimed stem cells.

It will be appreciated that an isolated population of primed stem cells (e.g., MSCs) can have an anti-inflammatory phenotype that is marked by any one or combination of the foregoing structural and/or functional effects.

Another aspect of the present disclosure can include a conditioned medium that is produced by culturing, contacting, or exposing stem cells (e.g., MSCs) to priming media of the present disclosure for a period of time and then collecting the supernatant, or conditioned medium, after the culture period. Advantageously, a conditioned medium of the present disclosure can include increased levels of immune modulatory mediators (e.g., IDO) as compared to conditioned medium prepared from unprimed stem cells, as well as other components, such as exosomes from the primed stem cells.

Uses

In another aspect, methods of use of an isolated population of primed stem cells and/or conditioned medium prepared from the isolated population of primed stem cells are described herein. In one example, a population of primed stem cells and/or conditioned medium prepared from the isolated population of primed stem cells may be used for the production of a pharmaceutical composition for the use in subjects in need of treatment, e.g., a subject that has, or is at risk of developing, a systemic inflammatory or autoinflammatory disease or disorder.

Compositions comprising a population of primed stem cells and/or conditioned medium prepared from the isolated population of primed stem cells as disclosed herein have a variety of uses in clinical therapy, research, development, and commercial purposes. For therapeutic purposes, for example, a population of primed stem cells and/or conditioned medium prepared from the isolated population of primed stem cells as disclosed herein may be administered to treat a subject that has, or is at risk of developing, a systemic inflammatory or autoinflammatory disease or disorder, such as an interferon-regulated autoimmune disease. In some instances, a population of primed stem cells, their cell membrane exosomes, and/or conditioned medium prepared from the isolated population of primed stem cells as disclosed herein can be administered to a subject not only help restore function to damaged or otherwise unhealthy tissues, but also to facilitate remodeling of the damaged tissues.

For research and development purposes, a population of primed stem cells and/or conditioned medium prepared from the isolated population of primed stem cells as disclosed herein may be used for development and studies of disease models specifically related to systemic inflammatory or autoinflammatory diseases or disorders.

One aspect of the present disclosure can include a method for treating a subject that has, is suspected of having, or is at risk of developing, a systemic inflammatory or autoinflammatory disease or disorder. In some instances, the systemic inflammatory or autoinflammatory disease or disorder can be an interferon-regulated autoimmune disease, such as an interferon-regulated autoimmune disease that is regulated by abnormal activation of a Type I IFN-mediated pathway and a Type II IFN-mediated pathway. Non-limiting examples of interferon-regulated autoimmune diseases treatable by the present disclosure can include SLE, Sjogren's syndrome, organ transplant rejection (e.g., graft-versus-host disease), adult or juvenile onset psoriatic arthritis, dermatomyositis, polymyositis, CNS vasculitis, scleroderma, inflammatory bowel disease, rheumatoid arthritis, and systemic sclerosis. Methods for determining whether a subject has, is suspected of having, or is at risk of developing any of the foregoing interferon-regulated autoimmune diseases are known in the art.

The method can comprise administering to the subject a therapeutically effective amount of primed stem cells; that is, stem cells having an anti-inflammatory phenotype marked by increased expression of one or more anti-inflammatory or immune modulatory mediators as compared to unprimed stem cells. Advantageously, administering the therapeutically effective amount of primed stem cells can have any one or combination of the following cellular effects: (1) increased expression of IDO, CD274, CD146 and PDGFRB and decreased expression of CX3CL1; (2) increased production of cytotoxic T cells; (3) increased production of Th1 cells; (4) increased production of CD4⁺ T cells; (5) increased production of NK cells; (6) suppressed production of Th17 cells; (7) suppressed production of double-stranded DNA; (8) suppressed production of BAFF; and (9) decreased production of IL-17.

It has been surprisingly found by the inventors of the present application that administering a single dose of primed stem cells (e.g., primed MSCs) results in the foregoing cellular effects (e.g., cellular effects (1)-(9)) for an extended period of time. Accordingly, in some instances, the therapeutically effective amount of primed stem cells (e.g., primed MSCs) can be administered as a single dose to a subject, whereafter any one or combination of cellular effects (1)-(9) can persist for an extended period of time including, but not limited to, about 1-12 months, about 1-2 months, about 1-3 months, about 1-4 months, about 1-5 months, about 1-6 months, about 1-7 months, about 1-8 months, about 1-9 months (e.g., about 9 months), about 1-10 months, about 1-11 months, or about 1 year.

In one example, a human subject that has, is suspected of having, or is at risk of having SLE can be treated according to the methods described herein. SLE is an autoimmune disease that is characterized by abnormalities in cellular and humoral auto-immunity. Pathogenic T-cells and B-cells recognize self-antigens resulting in immune hyperactivity and autoantibody production that culminates in a multisystem chronic inflammatory disease. Clinical presentations of SLE are highly heterogeneous, ranging from mild systemic inflammation that affects skin or joints to severe organ damage (brain, kidney, etc.). Unfortunately, there is still no uniformly effective treatment targeting both cellular and humoral autoimmunity for SLE. Therapies targeting components of cellular or humoral immune system fails to induce sustained remission in disease activity in multicenter clinical trials.

Advantageously, a therapeutically effective amount of primed stem cells can be administered to a human that has, is suspected of having, or is at risk of having SLE. Clinical methods for determining whether a subject is suspected of having, or is at risk of having, a systemic inflammatory or autoinflammatory disease or condition, are known in the art. In one example, the primed stem cells can comprise primed MSCs that have been contacted or cultured with a six-component priming medium. The priming medium can be administered alone or as a pharmaceutical composition (described below). Advantageously, the administered primed MSCs can exhibit any one or combination of cellular effects (1)-(9) (above), which, in turn, have a positive regulatory effect on the immune cells known to be responsible for dysregulation of the Type I and Type II interferon pathways in SLE. Specifically, primed MSCs of the present disclosure can expand and activate CD4+ T cells and CD8+ T-cells, NK cells and T regulatory (Treg) cells while suppressing of dendritic cells (DC) and B-cells via control of production and activity of Type I (-α, -β) and II interferons (-γ).

In another example, since primed stem cells (e.g., MSCs) can advantageously prevent or mitigate kidney sclerosis as well as inflammation, a therapeutically effective amount of primed stem cells (e.g., primed MSCs) can be administered to a subject before, during, or after an organ (e.g., kidney, liver, lung) transplantation procedure. There are many ways that primed stem cells (e.g., MSCs) can be used in the setting of organ transplantation. The quality of organs that are procured can vary greatly depending on the age and health of the donor, the amount of time the organ is outside of the body (and the storage conditions), and characteristics within the recipient. Too many organs are thrown out because they do not meet the surgeon's criteria for transplantation—often due to the ischemic time incurred within the donor post brain death or transit time outside of the donor but en route to the recipient. This is obviously a devastating loss each time and is particularly important for organs with very short out-of-body timelines. Currently, physicians perfuse organs (outside of any body) with specific cocktails of media during transport to reduce endothelial inflammation/activation and yield better prognosis for the patient; cocktails including using stem cells or stem cell by-products are being actively investigated. There is also work being done with treatment of recipients at (or near) time of transplant to help reduce delayed graft function (kidney) and ischemic caused inflammation/damage and/or to reduce requirement of toxic immunosuppressants via increasing tolerance (Treg) etc. Advantageously, primed stem cells (e.g., MSCs) have great potential in this area. This is because primed stem cells (e.g., MSCs) of the present disclosure have been found to reduce IFN-γ responses and promote T regulatory cells/tolerance, which can be leveraged in a variety of methods to support organ utilization, organ health, and success of organ transplantation.

In another aspect, the present disclosure can include a method for adoptive immunotherapy in a subject. Adoptive immunotherapy is a cell therapy that involves the removal of immune cells from a subject, the ex vivo processing (i.e., activation, purification and/or expansion of the cells) and the subsequent infusion of the resulting cells back into the same subject. Examples of adoptive immunotherapy methods include methods for producing and using LAK cells (Rosenberg U.S. Pat. No. 4,690,915), TIL cells (Rosenberg U.S. Pat. No. 5,126,132), cytotoxic T-cells (Cai, et al. U.S. Pat. No. 6,255,073; Celis, et al. U.S. Pat. No. 5,846,827), expanded tumor draining lymph node cells (Terman U.S. Pat. No. 6,251,385), various preparations of lymphocytes (Bell, et al. U.S. Pat. No. 6,194,207; Ochoa, et al. U.S. Pat. No. 5,443,983; Riddell, et al. U.S. Pat. No. 6,040,180; Babbitt, et al. U.S. Pat. No. 5,766,920; Bolton U.S. Pat. No. 6,204,058), CD8+ TIL cells (Figlin et al. (1997) Journal of Urology 158:740), CD4+ T-cells activated with anti-CD3 monoclonal antibody in the presence of IL-2 (Nishimura (1992) J. Immunol. 148:285), T-cells co-activated with anti-CD3 and anti-CD28 in the presence of IL-2 (Garlic et al. (1999) Journal of Immunotherapy 22:336) antigen-specific CD8+ CTL T-cells produced ex vivo and expanded with anti-CD3 and anti-CD28 monoclonal antibodies (mAb) in the presence of IL-2 (Oelke et al. (2000) Clinical Cancer Research 6:1997), and injection of irradiated autologous tumor cells admixed with Bacille Calmette-Guérin (BCG) to vaccinate subjects followed seven days later by recovery of draining lymph node T-cells which are activated with anti-CD3 mAb followed by expansion in IL-2 (Chang et al. (1997) Journal of Clinical Oncology 15:796).

In some instances, the method can comprise contacting an unprimed population of CAR-T cells and/or CAR-NK with a priming medium of the present disclosure for a time and under conditions sufficient to expand the CAR-T cells and/or CAR-NK cells and increase the cytotoxic activity of the CAR-T cells and/or CAR-NK cells. The primed CAR-T cells and/or CAR-NK cells can be created by an in vitro method of the present disclosure (e.g., using a four-component or six-component priming medium). A therapeutically effective dose of the primed CAR-T cells and/or CAR-NK cells can then be administered to a subject in need of adoptive immunotherapy (e.g., a subject having, suspected of having, or at risk of having a systemic inflammatory or autoinflammatory disease or disorder, such as SLE).

In some instances, suitable routes of administration for compositions comprising primed stem cells and/or conditioned medium prepared from the isolated population of primed cells can include intra-arterial or intravenous administration, parenteral administration, intrathecal administration, intraventricular administration, intraparenchymal administration, intracranial administration, intracisternal administration, intrastriatal administration, intranigral administration, intramuscular administration, intraspinal administration, subcutaneous administration, transdermal administration, pulmonary administration, nasal administration, rectal administration, and topical (including buccal and sublingual) administration.

A composition comprising an isolated population of primed stem cells and/or conditioned medium prepared from the isolated population of primed cells can be administered to a subject using an implantable device. Implantable devices and related technology are known in the art and are useful as delivery systems where a continuous or timed-release delivery of compositions delineated herein is desired. Additionally, an implantable device delivery system can be useful for targeting specific points of compound or composition delivery (e.g., localized sites, organs, bones, etc.) (Negrin et al., Biomaterials, 22(6):563 (2001)). Timed-release technology involving alternate delivery methods can also be used according to certain aspects of the present disclosure. For example, timed-release formulations based on polymer technologies, sustained-release techniques and encapsulation techniques (e.g., polymeric, liposomal) can also be used for delivery of the compositions delineated herein.

In some instances, therapeutic compositions can comprise an isolated population of primed stem cells alone and/or conditioned medium prepared from the isolated population of primed cells alone and/or an isolated population of primed stem cells and/or conditioned medium prepared from the isolated population of primed cells formulated as a pharmaceutical composition. Pharmaceutical compositions can comprise an effective number of primed stem cells and/or conditioned medium prepared from the isolated population of primed stem cells in combination with a pharmaceutically acceptable carrier (additive), additive, diluent or excipient (e.g., sterile saline, Hanks Balanced Salt Solution (HBSS) or Isolyte S, pH 7.4). For example, an isolated population of primed stem cells and/or conditioned medium prepared from the isolated population of primed stem cells as disclosed herein can be supplied in the form of a pharmaceutical composition, comprising an isotonic excipient prepared under sufficiently sterile conditions for human administration. For general principles in medicinal formulation, the reader is referred to Cell Therapy: Stem Cell Transplantation, Gene Therapy, and Cellular Immunotherapy, by G. Morstyn & W. Sheridan eds, Cambridge University Press, 1996; and Hematopoietic Stem Cell Therapy, E. D. Ball, J. Lister & P. Law, Churchill Livingstone, 2000. Choice of the cellular excipient and any accompanying elements of the composition will be adapted in accordance with the route and device used for administration.

Pharmaceutically acceptable carriers useful for formulating primed stem cells for administration to a subject are well known in the art and include, for example, aqueous solutions such as water or physiologically buffered saline or other solvents or vehicles such as glycols, glycerol, oils such as olive oil or injectable organic esters. A pharmaceutically acceptable carrier can contain physiologically acceptable compounds that act, for example, to stabilize or to increase the absorption of the conjugate. Such physiologically acceptable compounds include, for example, carbohydrates, such as glucose, sucrose or dextrans, antioxidants, such as ascorbic acid or glutathione, chelating agents, low molecular weight proteins or other stabilizers or excipients.

In some instances, an isolated population of primed stem cells and/or conditioned medium prepared from the isolated population of primed stem cells can optionally be packaged in a suitable container with written instructions for a desired purpose, such as the reconstitution or thawing (if frozen) of the primed stem cells and/or conditioned medium prior to administration to a subject.

In some instances, a therapeutically effective amount of primed stem cells and/or conditioned medium prepared from the primed stem cells can be administered to a subject that is sufficient to produce a statistically significant, measurable change in at least one symptom associated with a systemic inflammatory or autoinflammatory disease or disorder. Determination of a therapeutically effective amount is well within the capability of those skilled in the art. Generally, a therapeutically effective amount can vary with the subject's history, age, condition, sex, as well as the severity and type of the medical condition in the subject, and administration of other pharmaceutically active agents.

A composition comprising an isolated population of primed stem cells and/or conditioned medium prepared from the isolated population of primed stem cells can be administered to the subject at the same time or at different times. When administered at different times, the compositions for administration to a subject can be administered within 5 minutes, 10 minutes, 20 minutes, 60 minutes, 2 hours, 3 hours, 4, hours, 8 hours, 12 hours, 24 hours of administration of the other. When compositions are administered in different pharmaceutical compositions, routes of administration can be different. In some instances, a subject is administered a composition comprising primed stem cells alone and/or as a pharmaceutical composition. In other instances, a subject is administered a composition comprising conditioned medium prepared from the isolated population of primed stem cells alone and/or as a pharmaceutical composition. In another aspect, a subject is administered a composition comprising an isolated population of primed stem cells mixed with conditioned medium prepared from the isolated population of primed stem cells. In another aspect, such compositions can be administered at substantially the same time, or subsequent to each other.

In other instances, an isolated population of primed stem cells and/or conditioned medium prepared from the isolated population of primed stem cells can be stored for later implantation/infusion. An isolated population of primed stem cells and/or conditioned medium prepared from the isolated population of primed stem cells may be divided into more than one aliquot or unit such that part of the population of primed stem cells and/or conditioned medium prepared from the isolated population of the primed stem cells is retained for later application while part is applied immediately to the subject. Moderate to long-term storage of all or part of the primed stem cells in a cell bank is also within the scope of the present disclosure, as disclosed in U.S. Patent Publication No. 2003/0054331, which is incorporated herein by reference. At the end of processing, the concentrated primed stem cells and/or conditioned medium prepared from the isolated population of primed stem cells may be loaded into a delivery device, such as a syringe, for placement into the recipient by any means known to one of ordinary skill in the art.

In some instances, a composition comprising an isolated population of primed stem cells and/or conditioned medium prepared from the isolated population of primed stem cells for administration to a subject can further comprise a pharmaceutically active agent (e.g., an immune modulating agent), such as those agents known in the art for treatment of systemic inflammatory or autoinflammatory diseases or disorders. As used herein, the term “immune modulating agent” can include pharmaceutical agents that can either activate or increase normal immune function or inhibit or interfere with normal immune function. Examples of immune modulating agents that are immunosuppressive, for example, can include agents that inhibit T-cell/B-cell costimulation pathways, such as agents that interfere with the coupling of T-cells and B-cells via the CTLA4 and B7 pathways, as disclosed in U.S. Pat. No. 7,094,874, which is incorporated herein by reference. The immune modulating agent can be administered in a formulation which is compatible with the route of administration and is administered to a subject at a dosage sufficient to achieve the desired therapeutic effect. In some instances, the dose may be calculated using actual body weight obtained just prior to the beginning of a treatment course. For the dosages calculated in this way, body surface area (m²) is calculated prior to the beginning of the treatment course using the Dubois method: m²=(wt kg^{0.425}×height cm^{0.725})×0.007184.

Toxicity and therapeutic efficacy of administration of a composition comprising an isolated population of primed stem cells and/or conditioned medium prepared from the isolated population of primed stem cells can be determined by standard pharmaceutical procedures in cell cultures or experimental animals, e.g., for determining the LD50 and the ED50. Compositions comprising an isolated population of primed stem cells and/or conditioned medium prepared from the isolated population of primed stem cells that exhibit large therapeutic indices are preferred. The amount of a composition comprising an isolated population of primed stem cells and/or conditioned medium prepared from the isolated population of primed stem cells can be tested using several well-established animal models. In some instances, data obtained from the cell culture assays and in animal model studies can be used in formulating a range of dosage for use in humans. The dosage of such compositions lies preferably within a range of circulating concentrations that include the ED50 with little or no toxicity. The dosage may vary within this range depending upon the dosage form employed and the route of administration utilized.

The therapeutically effective dose or amount of a composition comprising an isolated population of primed stem cells and/or conditioned medium prepared from the isolated population of primed stem cells can also be estimated initially from cell culture assays. Alternatively, the effects of any particular dosage can be monitored by a suitable bioassay.

With respect to duration and frequency of treatment, it is typical for skilled clinicians to monitor subjects in order to determine when the treatment is providing therapeutic benefit, and to determine whether to increase or decrease dosage, increase or decrease administration frequency, discontinue treatment, resume treatment or make other alteration to treatment regimen. The dosing schedule can vary from once a week to daily depending on a number of clinical factors, such as the subject's sensitivity to the primed stem cells and/or conditioned media from the primed stem cells. The desired dose can be administered at one time or divided into subdoses, e.g., 2-4 subdoses and administered over a period of time, e.g., at appropriate intervals through the day or other appropriate schedule. Such sub-doses can be administered as unit dosage forms. In some instances, administration is chronic, e.g., one or more doses daily over a period of weeks or months. Examples of dosing schedules are administration daily, twice daily, three times daily or four or more times daily over a period of 1 week, 2 weeks, 3 weeks, 4 weeks, 1 month, 2 months, 3 months, 4 months, 5 months, or 6 months or more.

In another aspect, a population of primed stem cells as disclosed herein may be genetically altered in order to introduce genes useful in suppressing inflammation (e.g., genes that encode functional activators or inhibitors of Type I and Type II interferon pathways, or genes that encode anti-inflammatory mediators). In some instances, a population of primed stem cells can be genetically modified to enhance survival, control proliferation, and the like. A population of primed stem cells as disclosed herein can be genetically altered by transfection or transduction with a suitable vector, homologous recombination, or other appropriate technique, so that they express a gene of interest. In one example, one or more primed stem cells can be transfected with genes encoding a telomerase catalytic component (TERT), typically under a heterologous promoter that increases telomerase expression beyond what occurs under the endogenous promoter (see International Patent Application WO 98/14592, which is incorporated herein by reference). In another example, a selectable marker is introduced to provide for greater purity of the population of primed stem cells. In another example, a population of primed stem cells may be genetically altered using vector containing supernatants over an 8-16 h period, and then exchanged into growth medium for 1-2 days. Genetically altered primed stem cells can be selected using a drug selection agent such as puromycin, G418, or blasticidin, and then recultured.

Many vectors useful for transferring exogenous genes into primed stem cells as disclosed herein are available. The vectors may be episomal, e.g., plasmids, virus derived vectors such as cytomegalovirus, adenovirus, etc., or may be integrated into the primed stem cell genome, through homologous recombination or random integration, e.g., retrovirus derived vectors such MMLV, HIV-1, ALV, etc. In some instances, combinations of retroviruses and an appropriate packaging cell line may also find use, where the capsid proteins will be functional for infecting the primed stem cells as disclosed herein. Usually, primed stem cells will be incubated for at least about 24 hours in a culture medium. In some instances, the primed stem cells are then allowed to grow in the culture medium for short intervals in some applications, e.g., 24-73 hours, or for at least two weeks, and may be allowed to grow for five weeks or more, before analysis. Commonly used retroviral vectors are “defective”, i.e., unable to produce viral proteins required for productive infection. Replication of the vector requires growth in the packaging cell line.

The host cell specificity of the retrovirus is determined by the envelope protein, env (p120). The envelope protein is provided by the packaging cell line. Envelope proteins are of at least three types, ecotropic, amphotropic and xenotropic. Retroviruses packaged with ecotropic envelope protein, e.g., MMLV, are capable of infecting most murine and rat cell types. Ecotropic packaging cell lines include BOSC23 (Pear et al. (1993) PNAS 90:8392-8396). Retroviruses bearing amphotropic envelope protein, e.g., 4070A, are capable of infecting most mammalian cell types, including human, dog and mouse. Amphotropic packaging cell lines include PAl2 (Miller et al. (1985) Mol. Cell. Biol. 5:431-437); PA317 (Miller et al. (1986) Mol. Cell. Biol. 6:2895-2902) GRIP (Danos et al. (1988) PNAS 85:6460-6464). Retroviruses packaged with xenotropic envelope protein, e.g., AKR env, are capable of infecting most mammalian cell types, except murine cells. In some instances, the vectors may include genes that must later be removed, e.g., using a recombinase system such as Cre/Lox, or the cells that express them destroyed, e.g., by including genes that allow selective toxicity such as herpesvirus TK, Bcl-Xs, etc.

Suitable inducible promoters are activated in a desired primed stem cell (target) type, either the transfected cell, or progeny thereof. By transcriptional activation, it is intended that transcription will be increased above basal levels in the target cell by at least about 100 fold, more usually by at least about 1000 fold. Various promoters are known that are induced in different cell types.

In another aspect, the primed stem cells can be used for the screening of potential therapeutic agents. Potential therapeutic agents can be applied to primed stem cells in culture at varying dosages, and the response of the primed stem cells monitored for various time periods. Physical and/or functional characteristics of the primed stem cells can be analyzed by observing, e.g., cell growth with microscopy. The induction of expression of new or increased levels of proteins, such as enzymes, receptors and other cell surface molecules, or of amino acids, can be analyzed with any technique known in the art which can identify the alteration of the level of such molecules. These techniques include immunohistochemistry using antibodies against such molecules, or biochemical analysis. Such biochemical analysis includes protein assays, enzymatic assays, receptor binding assays, enzyme-linked immunosorbant assays (ELISA), electrophoretic analysis, analysis with high performance liquid chromatography (HPLC), Western blots, and radioimmune assays (RIA). Nucleic acid analysis such as Northern blots can be used to examine the levels of mRNA coding for these molecules, or for enzymes which synthesize these molecules.

It is also possible to screen for potential drug side-effects on primed stem cells by testing for the effects of potential therapeutic agents on cell proliferation and function of primed stem cells.

The following examples are for the purpose of illustration only and are not intended to limit the scope of the claims, which are appended hereto.

### Example 1

Experiments were conducted to develop a MSC priming medium, referred to as “HXB-319” in Example 1, to control the inflammatory activity induced by activation of the Type I and Type II interferon pathways in multiple autoimmune diseases, such as SLE, Sjogren's disease, systemic sclerosis, graft-versus-host disease, inflammatory bowel disease, multiple sclerosis, and psoriasiform arthritis. The term “HXB-319”, as used in Example 1, can also refer to MSCs that have been treated or primed with the MSC priming medium discovered in Example 1.

In Vitro Experiments Using Danger Signal Stimulation on Healthy Bone Marrow-Derived MSCs

Poly (I:C) Exposure of Human MSC Cultures Resulted in Anti-Inflammatory Response

The inventors used standard methods to isolate mixed MSC population from healthy donor and then treated MSCs on second passage with danger signals, such as Poly (I:C) (30 ng/ml), Lipopolysaccharide (LPS) (10 ug/ml), TNF-α (50 ng/ml), and IL-10 (25 ng/ml) over night (18 hours). Then the inventors changed their medium, rested them in serum, and collected them in 24 hours to check for secreted proteins. The protein arrays were performed using Ray Biotech protein arrays. The secreted proteins of primed and unprimed MSCs in the tissue culture media were analyzed using pathway analysis for biomarker detection (Ingenuity.com). The inventors identified five canonical pathways that are activated and three down-regulated after cells were exposed to Poly (I:C). The five pathways activated are:

(1) cytokine production known to be produced by macrophages and T-helper cells were upregulated: CSF-2 (16.5 fold), IL-13(3.7 fold), CCL4 (2.8 fold), IL-10 (2.5 fold) and TNF-α (2.15 fold) increased; while IL-10 (−4.3), CXCL1 (−5.4 fold), IL-3 (−1.6) fold decreased. While cytokine signaling pathways that regulate IL-17A and B, F, and interferon gamma pathways were down-regulated (NF kappa B complex, Interferon gamma, and IL-17 dimer and IL-17 receptor in combination regulate a chain of chemokines and cytokines in this pathway, such as CX3CL, CCL1, CCL4, CXCL6, CCL2, CCL11, CCL18, and IL1/IL6/TNF-α), cytokine signaling pathways that aid T helper cell differentiation were up-regulated (IFN-γ (1.6 fold), IL-4 (23 fold), IL-5 (3.1 fold), IL-6 (1.6 fold) IL-13 (3.7 fold), TGF-β (2.3 fold) and IL-10 (−4.3));

(2) cellular movement, hematopoiesis, immune cell trafficking pathways were down-regulated after poly (I:C) treatment. In this pathway analysis, the inventors saw that the important proteins that play a role in SLE pathogenesis, such as interferons and their regulators, such as IL-12, interferon gamma are down-regulated. Furthermore, a pro-inflammatory cytokine, IL-6, that works on T and B-cell activation together was remarkably suppressed;

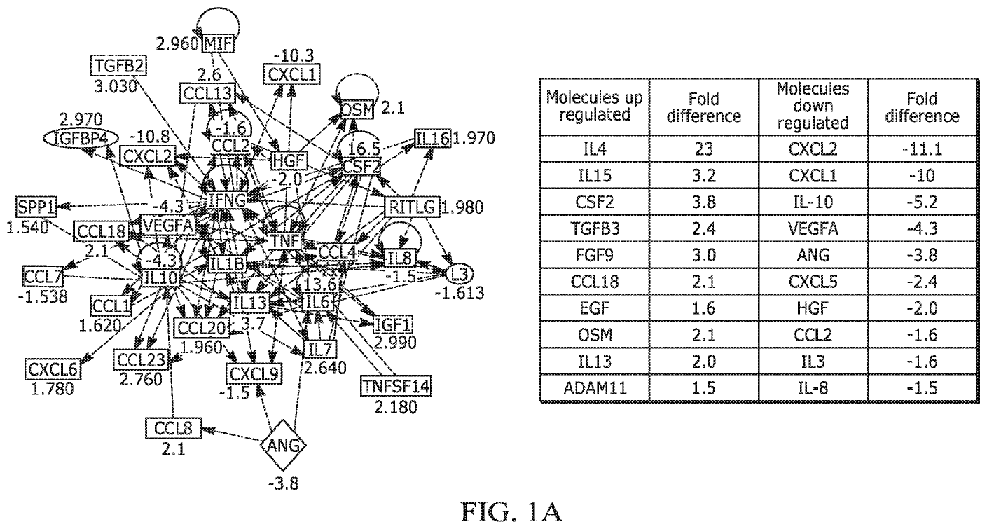

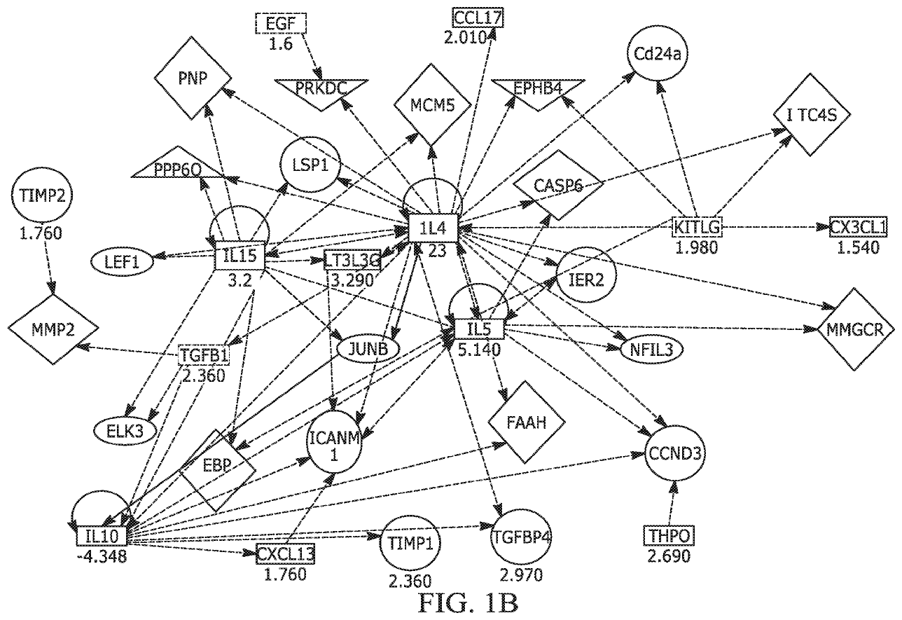

(3) cellular growth and proliferation, connective tissue development and function, lymphoid tissue structure and development network (FIG. 1A) showed remarkable suppression of CSF, IGF, HGF, FGF and LTA;

(4) the network (FIG. 1B) that involves TLR2/TLR4 activation that results with MAPK Erk1/2 activity and FGF, FGF6, -4, -7, IL-1, TNFRSF11B and CSF activation was suppressed after Poly (I:C) treatment; and

(5) MSCs primed by Poly(I:C) were capable of increasing IL-1β production, which induced CCR-8 ligand activation. CCR-8 is a biomarker identifying CD4 (+) memory cells enriched for Foxp3 (+) T-regulatory cells involved in delivery of immune suppression.

So the pathways that were down-regulated included:

(1) signaling pathways that play role in psoriasis;

(2) signaling pathways that play a role in atherosclerosis; and

(3) the arthritis signaling pathway (IL-18 was down-regulated by 11 fold, CXCL5 by 1.7 fold, and CXCL1 by 5 fold).

It was therefore surprisingly discovered that Poly (I:C) priming of MSCs can control the abnormally elevated INF-gamma related pathways in vitro and in vivo and control autoimmune disease activity regulated with interferons, such as in SLE.

MSC Cell Sorting Experiment after Poly (I:C) Exposure

Cell sorting experiments showed that Poly (I:C) exposure of human MSC cultures (considered a mixed population after bone marrow isolation) resulted in significant expansion of CD146+/pericyte-like MSC phenotype. Based on our observations, it was surprising to find that hMSCs change their surface marker configuration and acquire a pericytic phenotype in vitro when exposed to certain danger signals, such as inflammatory cytokines found in damaged tissue.

Pericyte-Like MSCS within Mixed MSC Cultures have Potent and Important Immune-Modulatory Activity

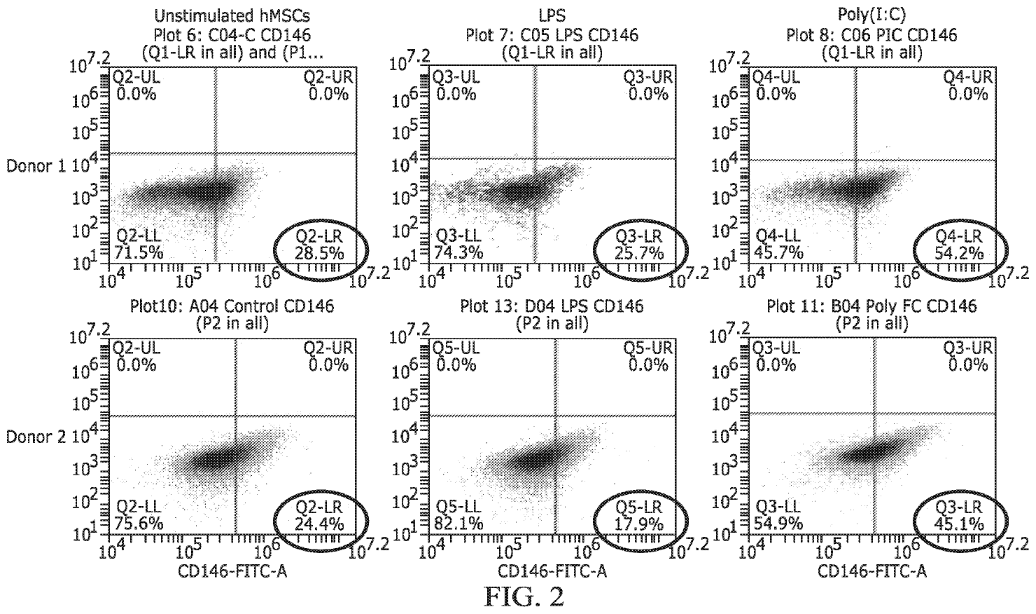

There is evidence that there is a direct relationship between the percentage of pericytes (defined as CD146+ and Lep-R+ cell surface receptors) within the unfractionated sample of MSCs, and the stimulus used to induce an immune-regulatory effect in vitro. By stimulating MSCs with Poly(I:C), it was found that the percentage of CD146+-MSCs significantly increased compared to unstimulated MSCs. In contrast, stimulating MSCs with LPS showed a decrease of CD146+-MSCs (FIG. 2).

Development of HXB-319

Cell Culture

The inventors validated the immune-stimulating effect of HXB-319 by comparing it with unprimed plain bone marrow-derived MSCs that are universally used in clinical trials currently.

Human Mesenchymal Stem Cell Culture Preparation

The hMSCs used in developing HXB-319 was obtained from the Hematopoietic Stem Cell Facility, which has been set up as a separate administrative entity under its own protocol (09-90-195). Bone marrow was harvested from normal human donors under a currently approved IRB protocol #09-90-195 at Case Western Reserve University (Cleveland, Ohio). Samples are de-identified as described in the implementation specifications of HIPAA regulation 45 CFR § 164.514 (b) (2) (i). The inventors performed experiments on mice and in vitro using 5 donors MSCs.

Human bone marrow-derived multipotent stromal cells (mesenchymal stem cells-MSCs) obtained from a healthy donor (2183 cell line) were cultured in log-growth phase in stem pro MSC SFM serum free media (Thermo Fisher Scientific) in Biospherix, a sterile conditioned environment at 37° C. in a humidified atmosphere and 5% CO₂. For experiments, cells were starved and maintained in stem pro basal medium for 48 hours before treatment with perturbation matrix.

RNA Extraction and PCR Analysis

Total cell mRNA was extracted using the MagMax RNA isolation kit (Thermo Fisher Scientific) according to manufacturer instructions. Nanodrop was used to measure the absorption at 260/280 nm to assess the quality and quantity of the collected RNA. Subsequently, the RNA was transcribed to first strand cDNA using the High capacity cDNA Reverse transcription kit (Thermo Fisher Scientific) for gene expression analysis. A customized open array gene chip was generated with 53 candidate genes and 3 house-keeping genes. Real time polymerase chain reaction (qPCR) was performed with SYBR® Green I master mix using QuantStudio (Thermo Fisher Scientific). The relative expression of the transcripts (RQ) was calculated using the 2 CT method.

Drug Matrix and Design of Experiment (DoE) Based Process Design

The experimental D-optimal designs are generated in software (Umetrics MODDE) and executed through a 96-well format on a Freedom Evo150 liquid handling robot (TECAN, CH).

The inventors picked the following 12 factors: Poly (I:C), TNF-α, IL-1β, IL-17, IFN-α, IFN-γ, PDGF-BB, Ascorbate 2 Phosphate, Endothelial growth factor (EGF), TGF-β, Vitamin D3). Then the inventors prepared simultaneous testing using a 96-run design, testing application. All cell culture, including manual preparatory expansion and seeding as well as robotic manipulations were contained in a modular X-Vivo system (Biospherix, NY, USA) providing Process Analytical Technology (PAT) of the cell culture conditions (affording % N₂, % CO₂, % CO₂, and temperature control). For each experimental condition, a total of 53 custom gene expression data are obtained using OpenArray technology (Life Technologies, Quantstudio Flex12). Mathematical modeling of the culture space is performed using the QPCR data within the MODDE software using multivariate tools for minimization of variance and model fitting. This process identified main effects and response parameters, and furthermore allowed us for optimization queries for interested gene amplification, such as anti-inflammatory cytokines or perivascular pericyte-like gene expression of MSCs. Implementing this process, the inventors defined a 6 component combination condition that maximizes IDO, CD274, CD146 and PDGFRB expression and minimizes expression for CX3CL1 and PDL1.

A series of complex multidimensional analyses were performed by Trailhead Biosystems (Cleveland, Ohio) analyzing 53 candidate gene targets based on the genes that were involved in the pathway analysis experiments performed and were found to have important roles in interferon activated signaling pathways. These were: ANG, BCL2, C3AR1, C5AR1, CCL17, CCL2, CCL5, CD274, CD36, CD44, CD55, CD59, CSF2, CSPG4, CX3CL1, CXCL1, CXCL12, CXCL3, CXCR4, ENG, ENTPD1, HSPA5, IDO1, IFNG, IL10, IL12A, IL17A, IL2, IL4, IL6, IL8, IRF3, LAMA4, LEPR, LIF, MCAM, NES, NGFR, NTSE, PDGFRB, PPBP, PTGS1, PTGS2, RGS5, SEMA3A, TGFB1, THY1, TLR3, TLR4, TLR7, TLR9, TNFAIP6 and VEGFA. These gene complex expression levels were normalized over 3 house-keeping genes (GAPDH, YWHAZ, TBP) in bone marrow-derived mesenchymal stem cells derived from healthy donor MSC lines using the following cytokines and concentrations: TNF-α (20 ng/ml), IFN-α (20 ng/ml), IFN-β (10 ng/ml), PDGF-BB (10 ng/ml), IFN-γ (100 ng/ml), IL-1β (10 ng/ml), IL-17A (50 ng/ml), Ascorbic acid 2 phosphate (200 uM), Poly I:C (1 ug/ml), TGF-β (10 ng/ml), EGF (20 ng/ml) and Vitamin D3 (10 ng/ml).

Using the DoE approach, a complex multidimensional experiment was created consisting of 96 conditions with simultaneous interrogation of 12 effectors in different combinations and concentrations. Bone marrow-derived MSCs obtained from healthy donor were seeded into a 96-well plate (12,000 cells/well) and analyzed at 6, 12, 18, 24 hours following drug matrix treatment by qPCR and exported to the MODDE software platform for further analysis. This generated more than 5000 data points. Each response was fitted onto a statistical regression model for analyzing how different perturbations affect gene expression. The inventors then extracted optimized conditions for maximizing gene expression of IDO1, PDGFRB, LEPR, IL-10, MCAM (CD146) and CD274 (PDL-1) in combination with minimizing CX3CL1 (Fractalkine activity). The resulted MSC population expressed high levels of MSC characteristic cytokines assuring they did not differentiate into a different cell kind. These were CD44, CD59, CD73, CD90, CD105, PDGFRB, TNFAIP6 and CD146. The MSC population after supercharging expressed higher levels of TLR-3 and TLR-4, IL6, IL8, IL12A, CCL2, CCL5 (RANTES), GMCSF, CXCL1, CXCL12, CXCL3 and ENG (Table 1).

| GENE | EXP VALUE | PREDICTED MAX | % OF MAX |
|---|---|---|---|
| ANG | 353.746 | 585.367 | 60.43 |
| BCL2 | 224.929 | 301.193 | 74.68 |
| C3AR1 | 8261.2 | 9976.66 | 82.81 |
| C5AR1 | 141.184 | 436.218 | 32.37 |
| CCL2 | 6530.2 | 8571.01 | 76.19 |
| CCL5 | 355777 | 582844 | 61.04 |
| CD274 | 11010.3 | 11145.2 | 98.79 |
| CD35 | 29.8773 | 64.1532 | 46.57 |
| CD44 | 107093 | 116555 | 91.88 |
| CD55 | 8176.45 | 8601.18 | 95.06 |
| CD59 | 31178.6 | 39176.4 | 79.59 |
| CSF2 | 9674.64 | 10408.1 | 92.95 |
| CSPG4 | 1513.73 | 3743.71 | 40.43 |
| CX3CL1 | 1380.06 | 1746.42 | 79.02 |
| CXCL1 | 554209 | 637764 | 86.90 |
| CXCL12 | 5650.58 | 11840.7 | 47.72 |
| CXCL3 | 159369 | 197309 | 80.77 |
| CXCR4 | −5.70846 | 70.5659 | −8.09 |
| ENG | 11129.6 | 13896.5 | 80.09 |
| ENTPD1 | 3.97101 | 34.1359 | 11.63 |
| HSPAS | 42837.3 | 50103.6 | 85.50 |
| IDO1 | 45093.4 | 44670.3 | 100.95 |
| IL10 | 0.0348632 | 5.80917 | 0.60 |
| IL12A | 836.17 | 1351.7 | 61.86 |
| IL4 | 1.59565 | 14.8789 | 10.72 |
| IL6 | 245130 | 385413 | 63.60 |
| IL8 | 1180000 | 1453470 | 81.19 |
| IRF3 | 3723.11 | 4760.41 | 78.21 |
| LAMA4 | 1970.52 | 2986.45 | 65.98 |
| LEPR | 1712.22 | 1772.01 | 96.63 |
| LIF | 85.2924 | 5929.44 | 1.44 |
| MCAM | 2173.58 | 3723.74 | 58.37 |
| NES | 61.1177 | 474.635 | 12.88 |
| NGFR | 121.841 | 271.264 | 44.92 |
| NT5E | 13819.1 | 26808.3 | 51.55 |
| PDGFRB | 17055.4 | 27689.6 | 61.59 |
| PPBP | −2.41581 | 60.0808 | −4.02 |
| PTGS1 | 3112.55 | 11821.5 | 26.33 |
| PTGS2 | 21110.1 | 32974.9 | 64.02 |
| RG55 | 1.87003 | 30.6069 | 6.11 |
| SEMA3A | 197.108 | 610.577 | 32.28 |
| TGFB1 | 9287.37 | 17773.7 | 52.25 |
| THY1 | 12205 | 22420.6 | 54.44 |
| TLR3 | 5293.54 | 7338.27 | 72.14 |
| TLR4 | 1489.2 | 1667.03 | 89.33 |
| TLR7 | 10.8914 | 42.3348 | 25.73 |
| TLR9 | 38.0195 | 108.499 | 35.04 |
| TMFA1P6 | 264697 | 271420 | 97.52 |
| VEGFA | 4725.96 | 29587.5 | 15.97 |

Validation of the Priming Technology Using Commercially Available Bone Marrow Derived MSCs Such as Rooster Bioscience®, and Different Bone Marrow Donors

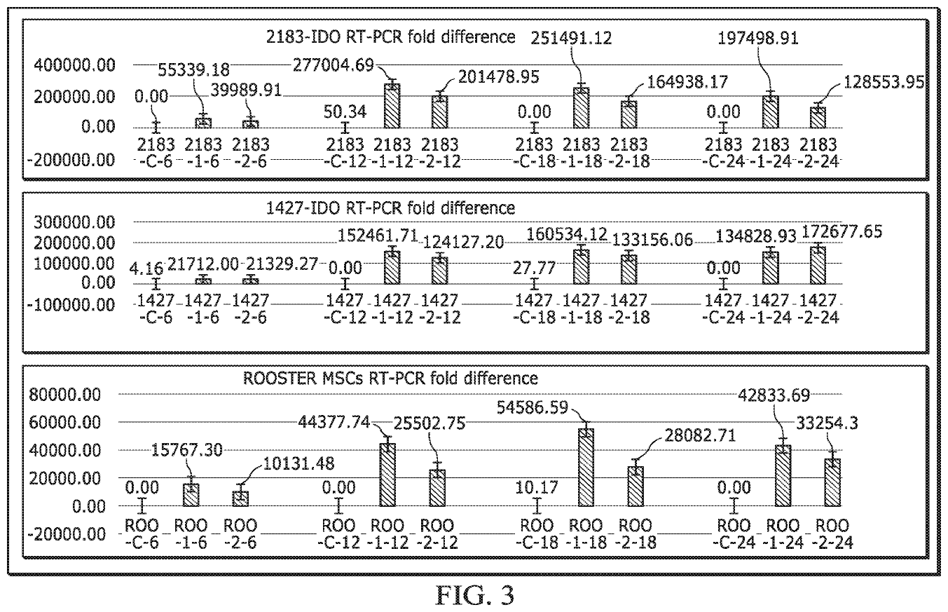

Using the same methodology described above, the inventors tested the priming method on different source of cells. Cells were cultured using the same robotic cell culturing technology using the same culture conditions and priming technology (i.e., HXB-319). As an efficacy assay, the inventors checked IDO gene expression fold difference. The inventors collected and isolated RNA from the MSCs at different time points of priming period (6 hour, 12 hour, 18 hour and 24 hours) and observed the maximum IDO gene expression across three unrelated primed MSC source (Numbered as 2183, 1427 healthy donor, at passage 3 and Rooster Bioscience cell product with unknown passage number). Condition 1 represents the HXB-319 condition while 2 is a control condition (FIG. 3). There was consistent fold increase in the primed MSC IDO gene expression across all sources of MSCs from three different health donor cell lines (FIG. 3).

Based on the foregoing experimental results, it was surprisingly discovered that HXB-319, which had the following formulation, could be used to induce or prime MSCs to obtain an anti-inflammatory phenotype to specifically regulate immune cellular activity and improve immune cell dysfunction after Type I and Type II interferon pathway induction (e.g., by autoimmunity):

(1) interferon-gamma, 100 ng/ml (eBioscience (BMS303));

(2) Poly (I:C), 1 ug/ml (Sigma Aldrich (9582-5 mg);

(3) tumor necrosis factor-α, 20 ng/ml (Peprotech (300-01A-50 ug));

(4) interleukin 1-β, 10 ng/ml, (Peprotech (200-01b-10 ug));

(5) interleukin-17-A, 50 ng/ml (Peprotech (200-17)); and

(6) ASC-AC-21, 200 μM (Sigma-Alrich (L4524-5MG)).

In Vitro and In Vivo Validation Experiments

In Vitro Bone Marrow Donor hMSC IDO Gene Expression and Kyruenine Enzymatic Activity in Tissue Culture Media

MSCs after passage three were seeded in the tissue culture flasks in triplicates. After confluency was achieved, the inventors removed the DMEM with 10% FBS and washed the cells three times with PBS and then treated with HXB-319 over 18 hours. Then, the inventors washed the HXB-319 and added serum-free DMEM to the culture plates and incubated in standard culture incubator. After 24 hours the inventors collected the MSCs and isolated RNA and then collected tissue culture media to measure secreted products. All results presented herein were replicated using at least three different MSC donors.

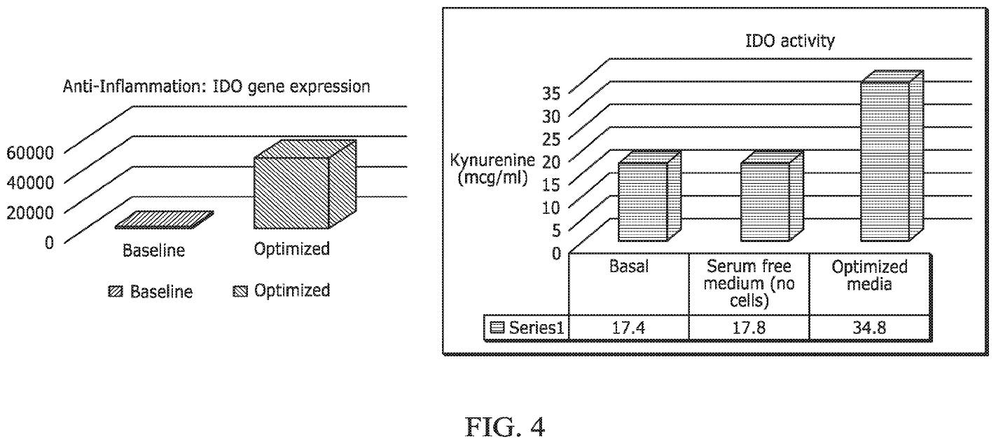

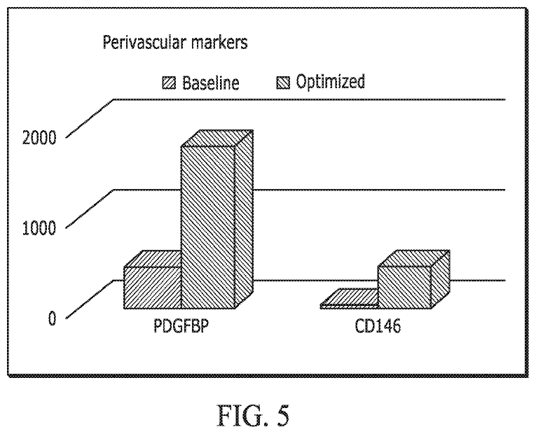

RT-PCR showed 50,000-fold elevation of IDO expression as compared to the untreated MSCs, and IDO assays showed significantly increased IDO enzymatic activity as measured by Kynurenine amount (mcg/ml) (FIG. 4). In addition, the perivascular MSC markers, such as PDGFBP and CD146 were significantly upregulated (FIG. 5).

Efficacy of HXB-319 in Pristane Induced Interferon Activation Mouse Model

For proof of concept the inventors used a validated and accepted mouse model in which type I interferon activation is induced over 4 weeks of time after intra peritoneal injection with 0.5 cc tetramethylpentadecane (pristane) (Freitas, E. C. et al., Clin Rheumatol, 2017. 36(11): p. 2403-2414; Gardet, A., et al., PLoS One, 2016. 11(10): p. e0164423; and Richards, H. B., et al., Kidney Int, 2001. 60(6): p. 2173-80).

The inventors obtained BALBC/cj female mice from Jackson Labs at 12-14 weeks of age and obtained 57BL/6 mice for control age and sex matched. The inventors performed 2 experiments using 2 sets of mice.

The inventors tested how mice injected with pristane responded to HXB-319, at acute phase of interferon activation and then in chronic phase of autoimmune disease activity that mimicked SLE. Type I interferon activation is induced over 4 weeks after intra peritoneal injection with 0.5 cc pristane. Subsequently interferon response genes (IRG) were expected to be activated generating almost an immune response profile that consist of further interferon activation. The activated pathways are: Type I IFN-α and -β, Type II IFN-γ, and proinflammatory cytokine elevation; IL-6, and IL-12. Collectively, these result in depletion of innate immune system responders: cytotoxic T cells (CD8+), T helper cells (CD4+) and NK cells.

Set 1 animals were used for acute experiments. About 4 weeks after pristane injection the inventors treated these mice with MSCs or HXB-319 MSCs in comparison to untreated mice (16 BALBC/j mice, 11 C57BL/6). A week after the treatment the inventors assessed the cellular repertoire in the peritoneal lavage fluid in an acute experiment.

Briefly: Day 0: Pristane injection to induce type I IFN activation; Day 28: MSCs or primed-MSCs (1×10⁶) were injected in 4 weeks; and Day 35: one week after MSC injection peritoneal lavage fluid and peripheral blood was collected from all mice.

Another set of animals set 2 (67 BALBc/j female and 20 C57BL/6) were injected at the same time points but kept for 270 days (˜9 months) before sacrifice, to read out the chronic effects of pristane model and outcomes of the HXB-319 treatment. The inventors collected, urine, blood and peritoneal lavage fluid and tissue from kidneys, liver, lungs, heart, lymph nodes, and spleen. The inventors also isolated splenocytes from spleen and performed a cell sorting experiments.

In our acute pristane injection mouse model studies surprisingly showed that MSCs primed with HXB-319 are safe, significantly effective and potent in ameliorating inflammation regulated by interferon activation in a lupus mouse model as opposed to non-primed MSCs that are currently in use by other groups in human clinical trials.

Peritoneal Lavage Fluid Cell Sorting

The inventors isolated the peritoneal lavage fluid and centrifuged and lyzed the red cells and fixed in 4% pfa. Then the inventors aliquot 3×10⁷ cells in two separate tubes for flow (Dendritic cells vs Lymphocytic panel) (see Table 2).

TABLE 2

| | Lymphocyte panel |
|---|---|
| DC panel antibodies | antibodies |
| Ly6G | CD3 |
| Ly6C | CD8 |
| B220 | B220 |
| CD11c | CD69 |
| CD11b | CD4 |
| PDCA-1 | NK1.1 |
| | PDL-1 |
| | CD11b |

In pristane induced mouse model, peritoneal lavage fluid cytotoxic T cell subset (CD4-CD8+) becomes lower than normal-WT due to interferon related inflammatory activity. Surprisingly, our primed MSCs are not only correcting lack of cytototoxic T cells (cd3+cd4−cd8+) but upregulating their numbers up to 7 fold (F=103.8, p=0.003), which shows significant potency as compared to unprimed MSCs (F=3.9, p=0.11) by multivariate analysis. In addition, the numbers of Th1 cells, CD4+ T cells (CD3+CD4+) up to are significantly upregulated up to 4 fold (p<0.05). These data were collected one week after our HXB-319 (primed MSC) treatment. Further, in vitro studies surprisingly showed that MSCs primed with HXB-319 express 50,000 times more indoleamine 2,3-Dioxygenase (IDO) as opposed to MSCs that are currently in clinical trials.

MSC Priming Using HXB-319 Showed In Vitro and In Vivo Evidence that Both Innate and Adaptive Immune System Cytotoxic Cells are Expanded, Activated and Modulated in Certain Targeted Signaling Pathways

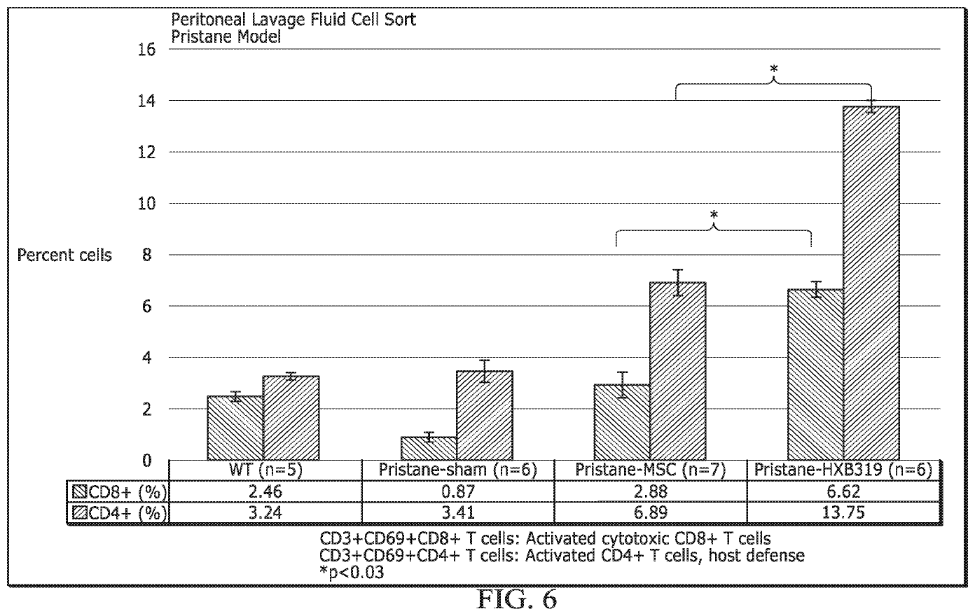

T cells: CD3+CD4+ T cells, CD3+CD8+ T cells, PD-L1+, CD25+CD4+& CD25+CD8+ T regulatory cells, all CD25+ cells were expanded significantly more than IL-2 stimulation of the same cells as per cells sorting experiments of in vivo (FIG. 6). And in in vitro experiments, CD-25+ was shown to be expressed on a subset of T cells, which have the ability of regulatory and suppressive capacities. CTLA-4+ T cells were also expanded in vivo and in vitro, which is an anti-inflammatory co-stimulatory molecule.

These cells are functionally activated as evident from cell surface receptor expression. CD-69+ is expressed significantly higher in the PBMCs co cultured with HXB-319 indicating activation of immune cells. CD69 is expressed by several subsets of tissue resident immune cells, including resident memory T (TRM) cells and gamma delta (γδ) T cells, and is therefore considered a marker of tissue retention. CD69 regulates the differentiation of regulatory T (Treg) cells as well as the secretion of IFN-γ, IL-17, and IL-22 (Eur. J. Immunol. 2017. 47: 946-953). CD56+ is also expressed in T cells and activation was remarkably increased in the absence of IL-2 with HXB-319 co-culture environment. It was shown that CD56+ T cells proliferated less vigorously but displayed enhanced natural cytotoxicity compared with CD56− T cells. CD56+ T cells released interferon-gamma (IFN-gamma) and interleukin-13 (IL-13), but not IL-10, upon TCR stimulation. Elevated proportions of CD56+ T cells expressed IFN-gamma, IL-4, and IL-13 within hours of activation. These acquired cytolytic and cytokine secretion activities of CD56+ T cells make them potential candidates for immunotherapy for immune-mediated diseases.

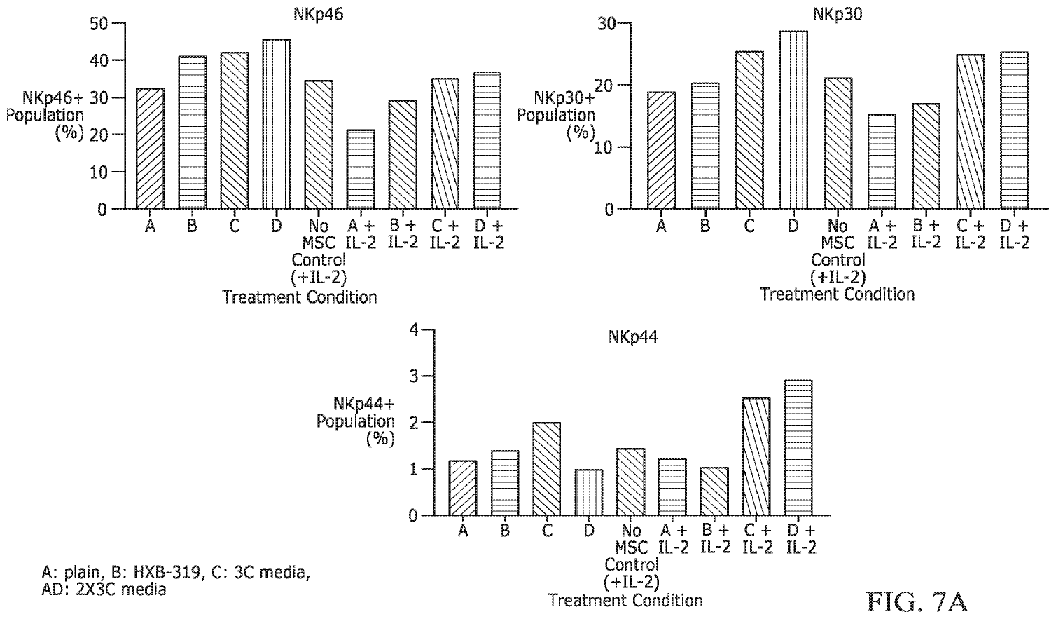

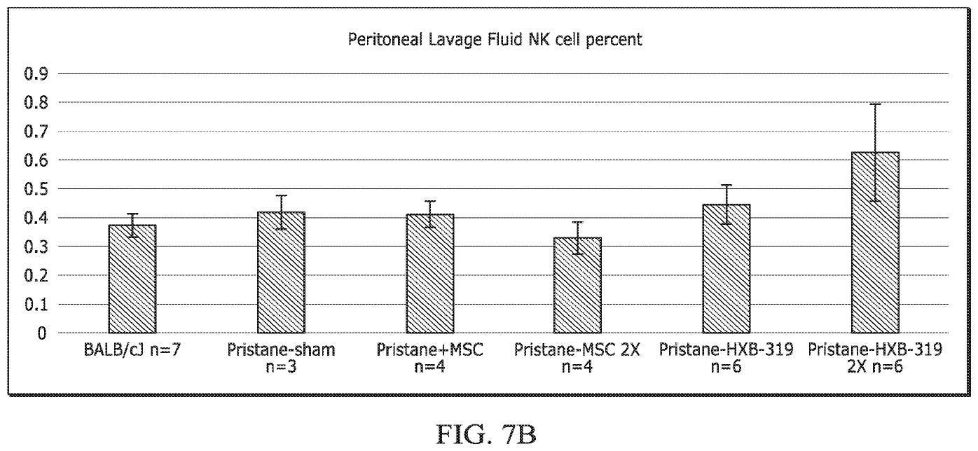

NK cells: CD25+NK cells were expanded in mice significantly in numbers when treated with HXB-319 as evidenced by the cell surface markers for NK cells, such as NKp46, NKp30, NKp44 in vitro. FIGS. 7A-B show the results of cell sorting experiments on PBMC after HXB-319 exposure and its 3-component derivate (referred to in Example 2 below as HXB-319-3) (FIG. 7A shows the following: B is HXB-319; C is HXB-319-3; and D is 2×HXB-319-3). CD-56 is the prototypic marker of NK cells are also remarkable expanded after HXB-319 treatment. FIG. 7B shows peritoneal lavage fluid NK cell percentage 9 months after HXB-319 and MSC treatments showing dose effect for HXB-319 and sustainable effect of HXB-319.

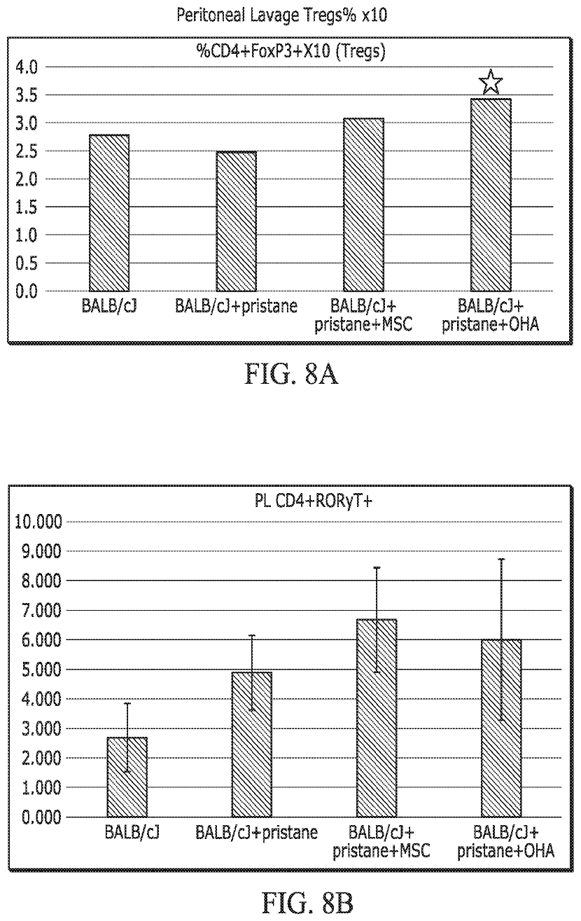

T regs: Regulatory T cells (Tregs) are a specialized subpopulation of T cells that act to suppress immune response, thereby maintaining homeostasis and self-tolerance. It has been shown that Tregs are able to inhibit T cell proliferation and cytokine production and play a critical role in preventing autoimmunity. It was surprisingly discovered that there was a remarkable increase in the CD4+ Tregs in vivo experiments after HXB-319 (OHA in FIG. 8A) treatment.

Th17 cells (RORgT+): Th17 cells are a specific subset of T helper lymphocytes determined by high secretion of IL-17 and other inflammatory cytokines.

Surprisingly, MSCs primed with HXB-319 controlled the activation and significantly suppressed the expansion of of Th17 cells, and this result was evidence of sustainable immune system modulation after 9 months of one-time injection with HXB-319 (1×10⁶ cells) over Th17 cells (FIG. 8B).

Th17 expression in splenocytes: CD8+RORgT+ cells (Th17 cells) in set 1: p<0.011, F=5.0, HXB-319, 1 million is significantly lower than healthy control and numerically lower than pristane-SLE mice, p<0.06, (3.33 untreated SLE control mean, 1.32 in HXB-319, coefficient of variation is high). While in chronic experiment CD8+RORgT+ cells in set 2: p<0.018, F=3.84, HXB-319, 1 million injection arm was significantly higher than pristane control.

Plasmacytoid dendritic cells pDCs: MSCs primed with HXB-319 were tested on interferon activation in vivo model and was shown to suppress the expansion/activation of splenic pDCs. The inventors observed similar results for the myeloid dendritic cells mDCs (Table 3):

TABLE 3
Chronic model set 2 Results of the splenocyte cell sorting 9 months
after MSC and HXB-319 treatments and their significance listed

| Splenocytes in | | | |
|---|---|---|---|
| BALBcj + Pristane model | Cell type | ANOVA F | p |
| PDCA-1+ | OHA 2.5 million X1 | F = 4.18, | p < 0.008 |
| Lymphoid DC cd11b/c+, | OHA 1 million X1 | NS | p = NS |
| B220+, Lyc- | | | |
| Myeloid DC cd11b+/c+ | OHA 2.5 million X1 | NS | p = NS |
| B220−, Lyc+ | | | |
| CD4+ FOXP3+ | OHA 1 million X1 | F = 3.77 | p < 0.016 |
| CD4+ PDL1+ | OHA 1 million X1 | F = 4.704 | p < 0.015 |
| CD8+ RORgT+ | OHA 1 million X1 | F = 3.84 | p < 0.018 |

Plasmacytoid dendritic cells are the main producers of type I IFNs and directly impact T cell responses through antigen presentation and represented with PDCA-1+ presence.

PDCA-1, is type II transmembrane glycoprotein with a molecular mass of 29-33 kD. It is predominantly expressed on Type I IFN-producing cells (IPCs) in naïve mice, but is up-regulated on most cell types following stimulation with type I IFNs and IFN-gamma. It is highly expressed on terminally differentiated normal plasmacytoid dendritic cells.

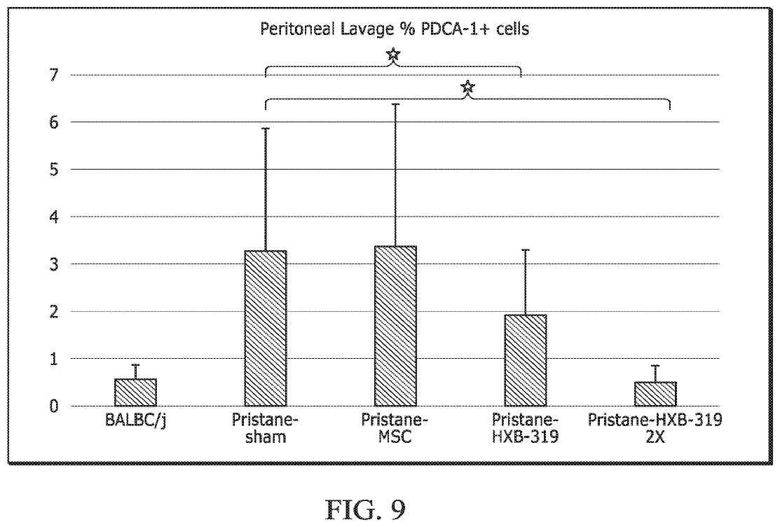

Surprisingly, it was discovered that PDCA-1+ splenocyte and peritoneal cavity cell frequency was significantly diminished when pristane model was treated with 2.5×10⁶ primed MSCs (HXB-319) as compared to the one million plain MSC (not primed) and 1 million HXB-319 treatments (Table 3, FIG. 9). PDCA-1+ also is a cell marker for plasmacytoid dendritic cells (pDCs) but could also be seen in the B cells. It is predominantly expressed on Type I IFN-producing cells (IPCs), but is up-regulated on most cell types following stimulation with type I IFNs and IFN-gamma.

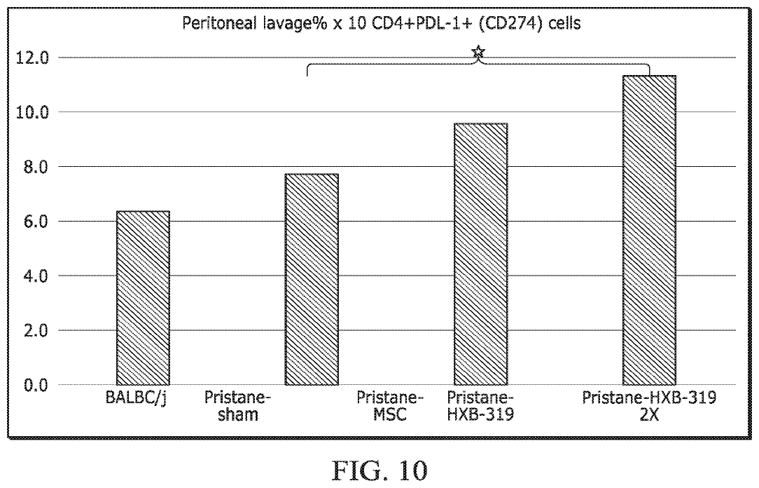

PD-L1+ cells (FIG. 10): PD-L1 plays an important role in immune regulation by binding to PD-1 expressed on effector T-cells to induce apoptosis or anergy in order to prevent autoimmune disease. This molecule is a check point molecule that needs to be in balance for normal immune defense. Its levels are low during inflammation and it is desirable to normalize or increase its levels while treating autoimmune diseases. PD-L1 is known to be CD274 and is considered an anti-inflammatory marker HXB-319 has a positive effect on the PD-L1 expressing cell percentage in both peritoneal lavage fluid and splenocyte cell percent count (FIG. 10) showing evidence for an anti-inflammatory effect. But, the level was not excessively high and there was no statistical difference in the levels of PD-L1 between wild-type untreated mice and pristane induced HXB-319 treated mice arms in the set 1 acute experiment.

CD4+PDL1+ cells in Set 1 (acute experiment; 1 week after cell treatment of 4 weeks post pristane induction) showed difference using ANOVA; untreated MSCs showed higher numbers than healthy controls, OHA ×1 million (HXB-319) onetime treatment was higher numerically.

CD4+PDL1+ cells in Set 2 (chronic experiment, 9 months after cell treatment of 4 weeks post pristane induction), difference using ANOVA, OHA treated MSCs ×1 (HXB-319)×1 million was significantly higher than healthy controls, and also higher numerically than SLE control mice (Pristane sham) (P<0.030, F=3.33).

Serum Interferon Levels

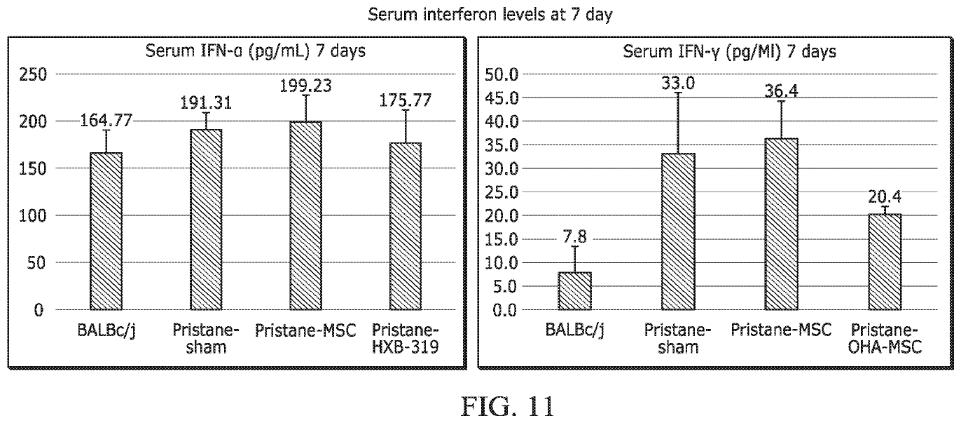

In acute pristane mouse model, one-time treatment of MSCs primed with HXB-319 (2×10⁶ cells) of pristane induced mice surprisingly suppressed both systemic levels of IFN-α (p<0.05) and IFN-γ (p<0.001) in serum (FIG. 11).

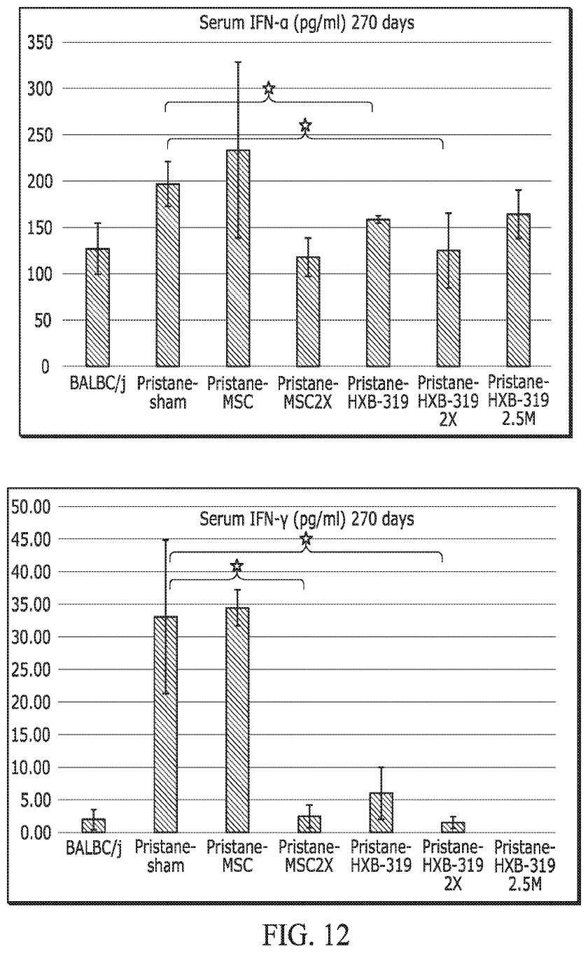

In chronic pristane mouse model, one time treatment of MSCs primed with HXB-319 (1× or 2×10⁶ cells) of pristane induced mice surprisingly showed sustainably suppressed systemic levels of IFN-α (p<0.05) and IFN-γ (p<0.001) (FIG. 12).

Moreover, it was surprisingly discovered that treatment with MSCs primed with HXB-319 successfully suppressed the type I and II IFN pathway activated inflammation in the acute or chronic pristane induced inflammatory disease, showing evidence that type I and II interferons activation pathways were controlled. In addition, there was significant improvement in the number of inflammatory cytokines after HXB-319 treatment.

Serum Cytokine Levels in Chronically Affected Pristane Model

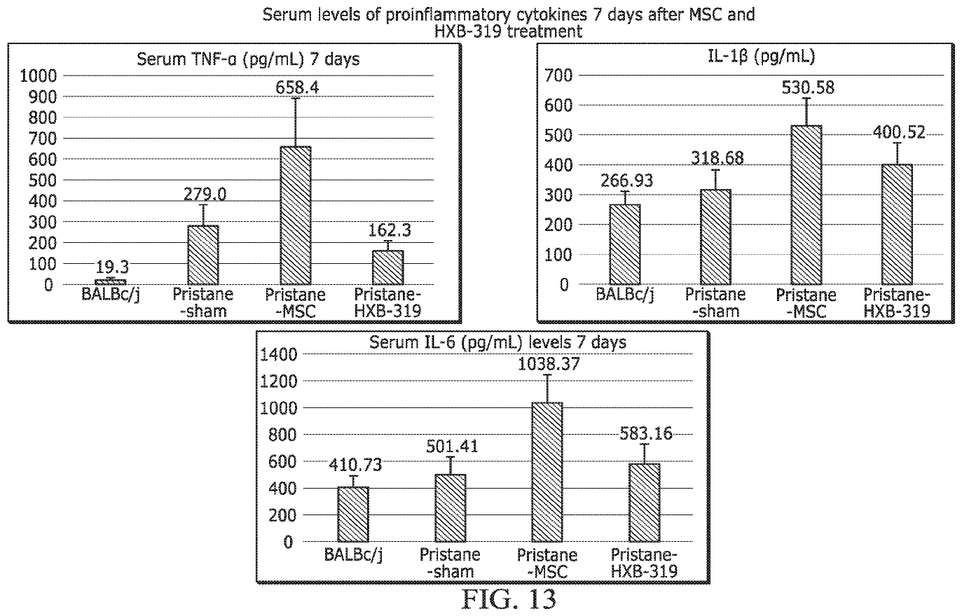

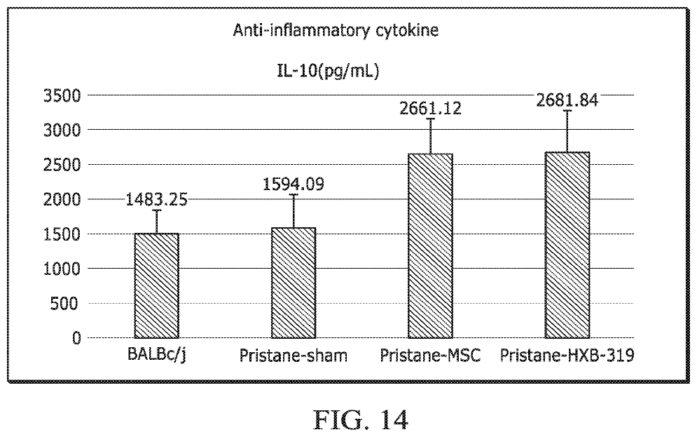

While cytokines such as TNF-α, IL-1β, and IL-6 are elevated after unprimed regular MSC treatments, MSCs primed with HXB-319 did not have a significant inflammatory cytokine elevation, thus raising no questions for increasing a cytokine storm (FIG. 13). In FIGS. 13-14, these cytokines were checked in the serum of pristane induced mice, one week after treatment with unprimed MSCs and MSCs primed with HXB-319.

Serum IL-17 Levels

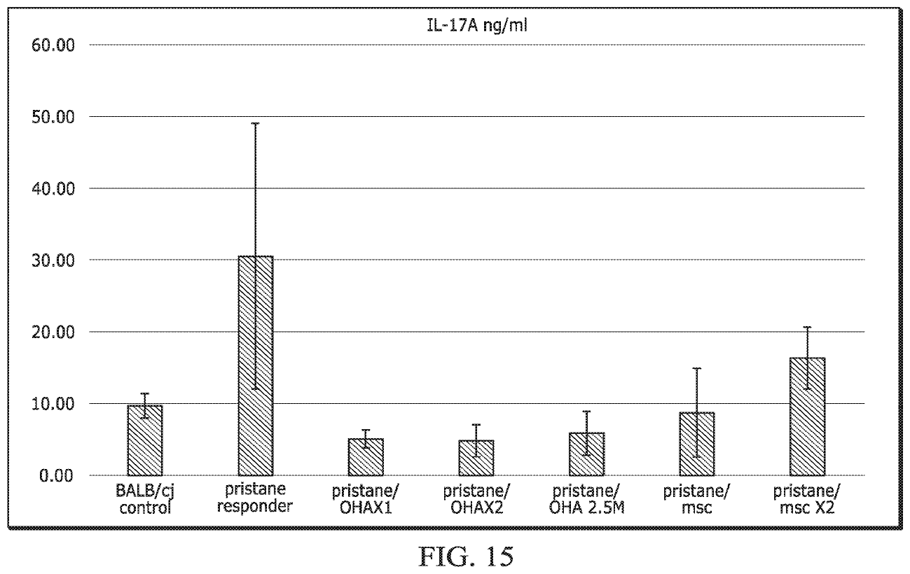

Serum levels of IL-17A was also checked in the mice serum after 270 days of pristane injection and treatment with unprimed MSCs and MSCs primed with HXB-319 (OHA in FIG. 15) surprisingly showed that HXB-319 treatment significantly decrease this cytokine level in a dose dependent way.

Double Stranded DNA Titer Change

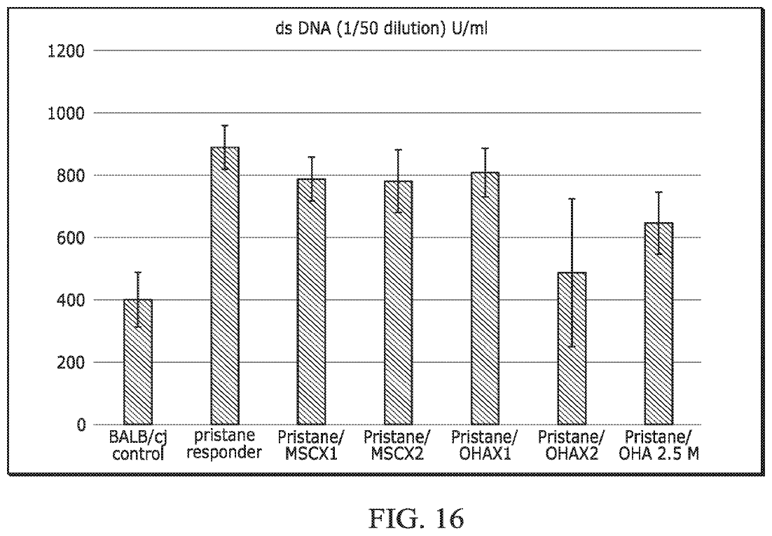

Double stranded DNA is a marker for nephritis and represents clinical improvement of SLE nephritis. Treatment with MSCs primed with HXB-319 showed significant control of this marker at higher doses. After ×2 use or above 2.5 million per mouse dose the effect was significant (FIG. 16).

Serum BAFF

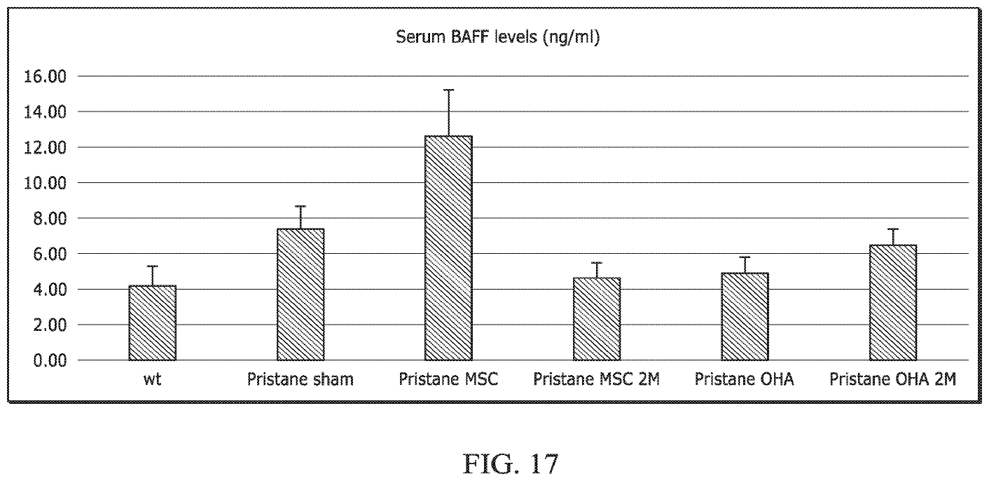

Serum levels of BAFF were also measured in the chronic experiment set (FIG. 17). The results showed successful tapering of BAFF levels using MSCs primed with HXB-319, which is a molecule that activates and stimulated B cell activation and is considered to be one of the major treatment targets in SLE. Belimumab the most recent FDA approved medication effect the B cell activation factor Blys as well.

Pathologic Evaluation and Scores for Chronic BALB/Cj Pristane Injection Experiments

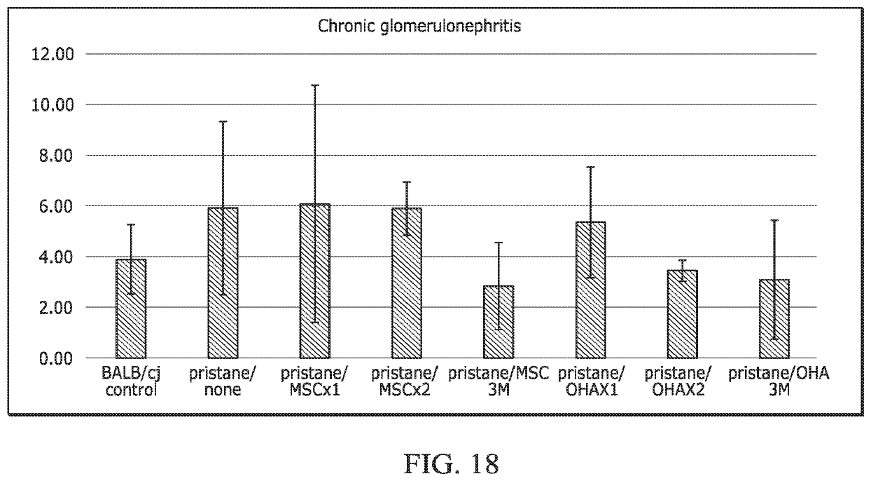

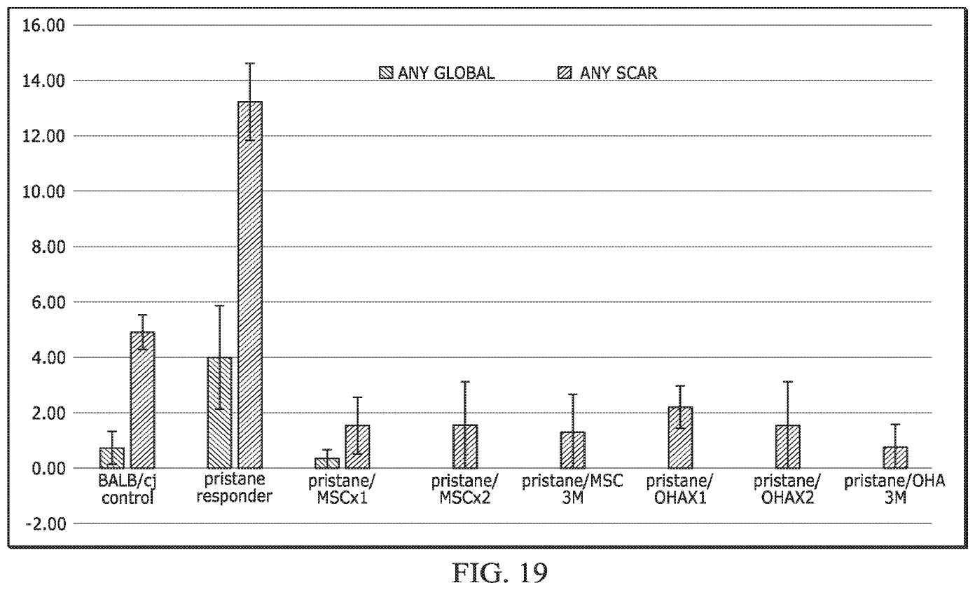

The inventors fixed all organs taken out of mice in formalin with 10% PBS. H and E and PAS staining was performed for all kidney biopsies and the inventors contracted an independent pathologist expert in renal pathology (FIGS. 18-19). Previously published renal pathology scores were used for numeric evaluation of inflammatory cells and sclerosis (Kidney Int 2009 September; 76(5):534-45, Epub 2009 Jul. 1). It was surprisingly discovered that MSCs primed with HXB-319 ameliorated glomerulosclerosis and global scarring in pristine induced BALBcj human SLE mouse model, and that MSCs primed with HXB-319 ameliorated dermatitis seen in pristine induced BALBcj human SLE mouse model.

In conclusion, through the experiments outlined in Example 1, it was surprisingly discovered that:

(1) MSCs primed with HXB-319 ameliorated serologic and cellular signs of interferon activation in pristine induced BALBcj interferon activation acute model and in chronic pristane induced human SLE mouse model;

(2) MSCs primed with HXB-319-3 were more efficacious than IDO gene expression, and in NK cell expansion and do not have same amount of IL-6 and IL-8 elevation seen after MSCs primed with HXB-319 in tissue culture in vitro. Thus, MSCs primed with HXB-319-3 can be used when there is cytokine storm seen in macrophage activation syndrome (MAS), a severe complication seen in many autoimmune diseases when immune activation is not controllable, including SLE active disease;

(3) MSCs primed with HXB-319 ameliorated glomerulosclerosis and global scarring in pristine induced BALBcj human SLE mouse model and, therefore, are preventative against end stage SLE nephritis;

(4) MSCs primed with HXB-319 ameliorated dermatitis seen in pristine induced BALBcj human SLE mouse model and, therefore, are preventative against skin manifestations of SLE;

(5) MSCs primed with HXB-319 improved NK, CD4+, and CD8+ T cell expansion and activity when there was interferon activation; and

(6) MSCs primed with HXB-319, or CCM isolated from MSCs primed with HXB-319 (which includes secreted products of the MSCs along with exosomes) when exposed to the CAR-T and CAR-NK cells can induce their expansion and cytotoxic activity and thereby be helpful to improve efficacy and patient outcomes. Cell therapy products such as CAR-T, CAR-NK and their further engineered variations, genetic variants, secreted product and exosomes can be primed with HXB-319 to improve their potency and longevity. Chimeric antigen receptor-modified T-cells (CART) or CAR-Natural Killer cells are immunotherapy and have been shown to induce remissions in some patients with chemotherapy refractory hematologic malignancies. HXB-319 can improve the efficacy of CAR-T/CAR-NK therapies and improve patient treatment outcomes by increasing the anti-inflammatory potency of CAR-T and CAR-NK cells.

### Example 2

Experiments were conducted to develop a MSC priming media, referred to as “HXB-319-3” in Example 2, to control the inflammatory activity induced by activation of the Type I and Type II interferon pathways in multiple autoimmune diseases, such as SLE, Sjogren's disease, systemic sclerosis, graft-versus-host disease, inflammatory bowel disease, multiple sclerosis, and psoriasiform arthritis. The term “HXB-319-3”, as used in Example 2, can also refer to MSCs that have been treated or primed with the MSC priming medium discovered in Example 2.

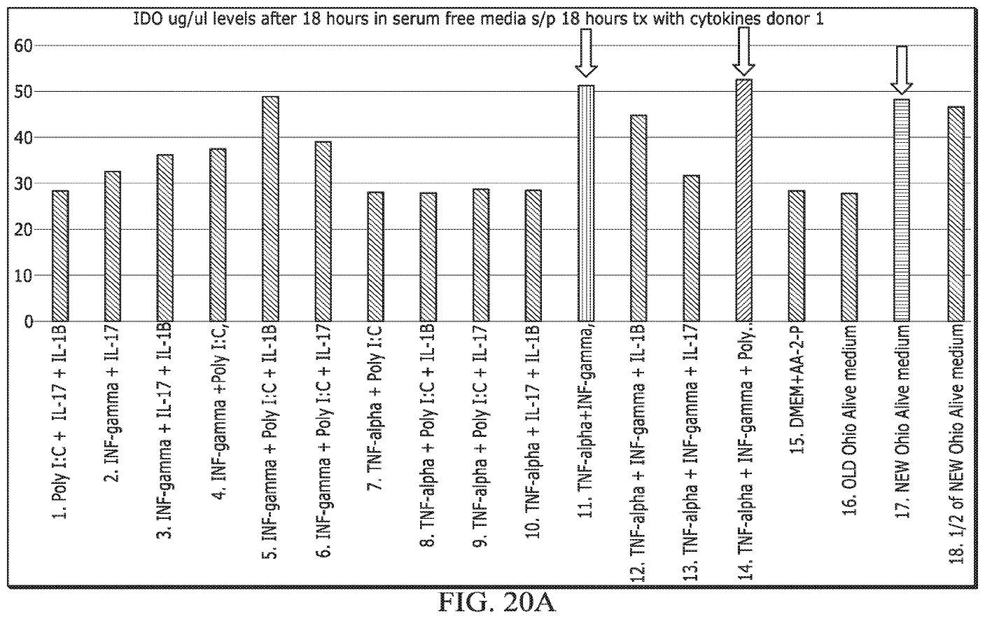

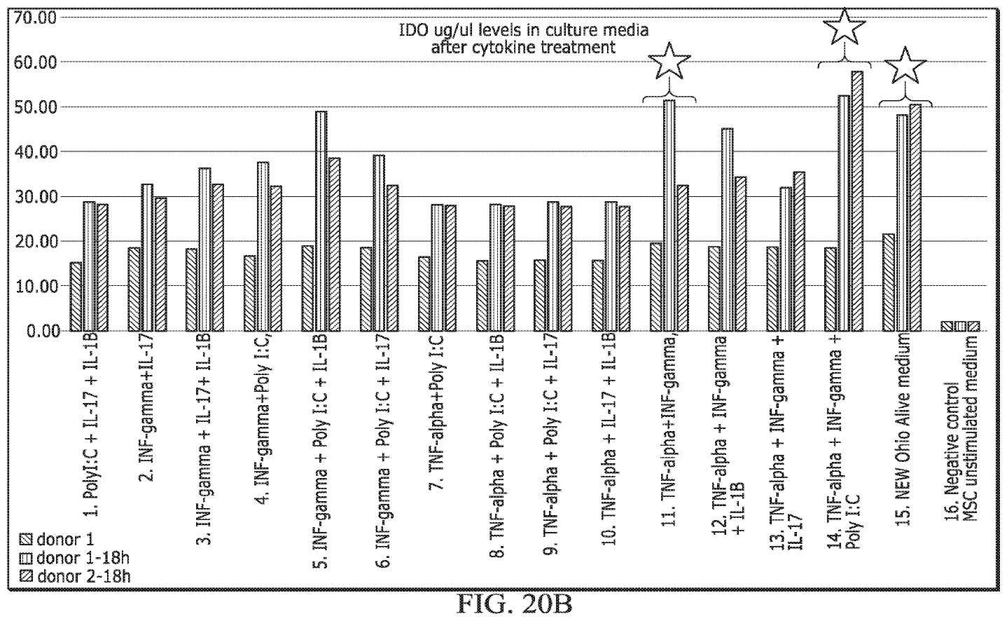

Similar to the experiments described in Example 1, the inventors tested different subsets of priming media (as compared to HXB-319) for maximizing gene expression of IDO1, IL-10, PDGFR, MCAM (CD146) and CD274 (PDL-1) in combination with minimizing CX3CL1 (Fractalkine) expression. As in Example 1, the inventors first assessed IDO expression and activity. ASC-AC-21, 200 μM (Sigma-Alrich (L4524-5MG)) was added to the serum free DMEM to all study arms. The different combinations of media components tested are indicated in FIGS. 20A-B.

The inventors used at least 5 different bone marrow donors' isolated MSCs and assessed the potency of the HXB-319-3 in vivo using IDO enzymatic assay and RT-PCR using the following genes: (CTLA4, IL-4, IL-6, IL-8, AKT, IL-17, TLR4, TLR-7, CX3CR3 and PDGF-R. The MSCs were grown and expanded as described in Example 1 and treated with serum free media that contained four components, i.e., IFN-γ (100 ng/ml), Poly (I:C) (1 μg/ml) and TNF-α (20 ng/ml) (collectively referred to as “HXB-319-3”), for 18 hours. At the end of 18 hours, cells were washed ×3 with PBS and then RNA was isolated using RNA isolation Qiagen kits. Then RT-PCR was performed using two replicates, and two sets of cells treated simultaneously were kept another 24 hours in serum free media and secreted products of the MSCs were collected for ELIZA. The results of two donor MSCs response in kynurenine activity (IDO) is shown in FIGS. 20A-B. Surprisingly, the results indicated that HXB-319-3 has the most potent anti-inflammatory effect and similar effects on anti-inflammatory gene expression in vitro.

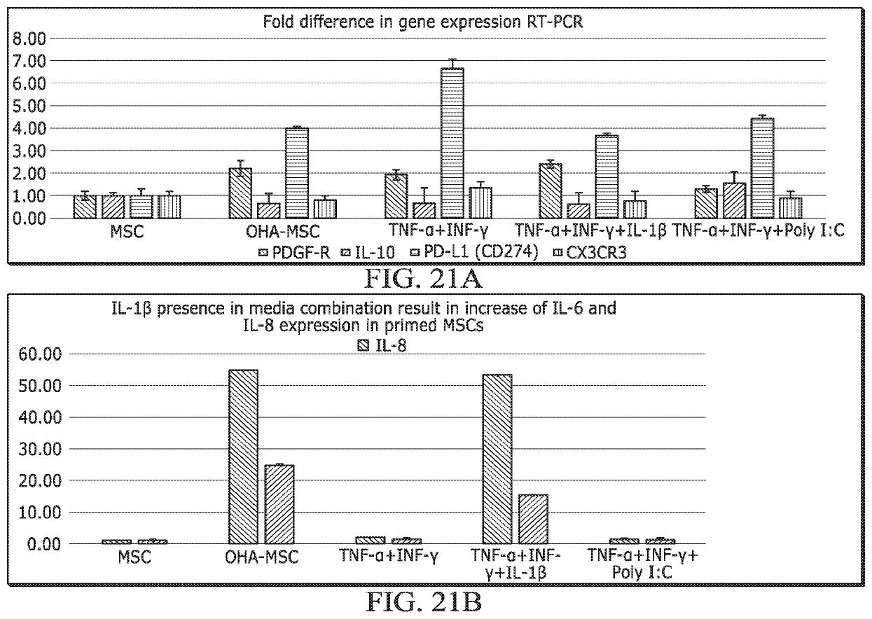

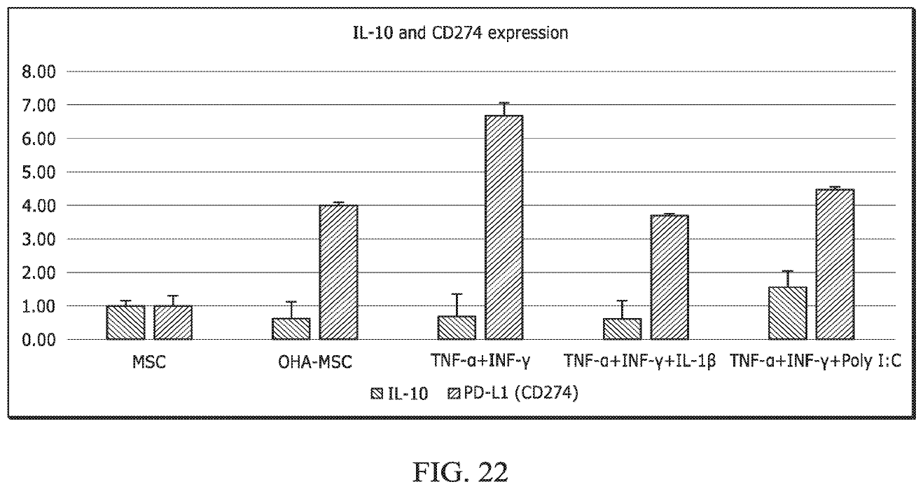

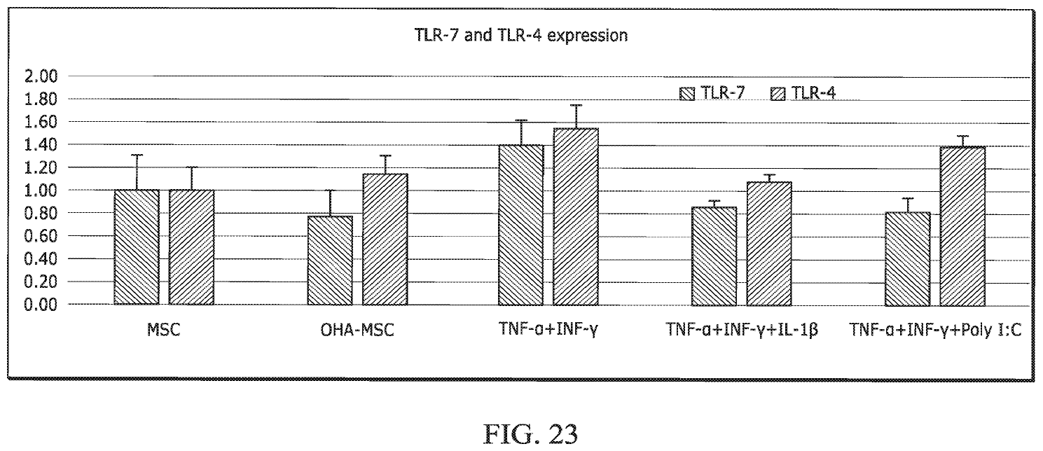

The inventors also performed RT-PCR on the cells treated with the study arms untreated MSCs: 15 (HXB-319); 11 (TNF-α+INF-γ); 12 (TNF-α+INF-γ+IL-1β); and 14 (TNF-α+INF-γ+Poly I:C); to verify the gene target expression were similar or better than those expressed in the HXB-319 treatment arm. The inventors particularly concentrated on the following genes: IDO1, IL-10, PDGRF and CD274 (PDL-1) in combination with minimizing CX3CL1 (FIGS. 21A-23).

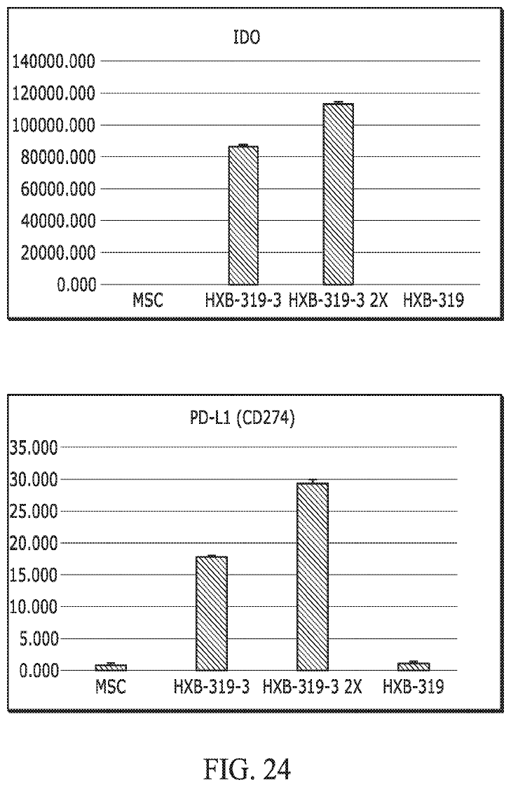

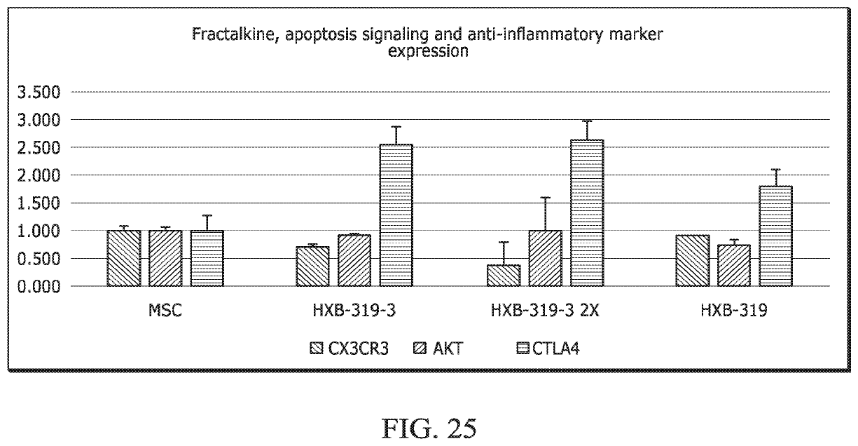

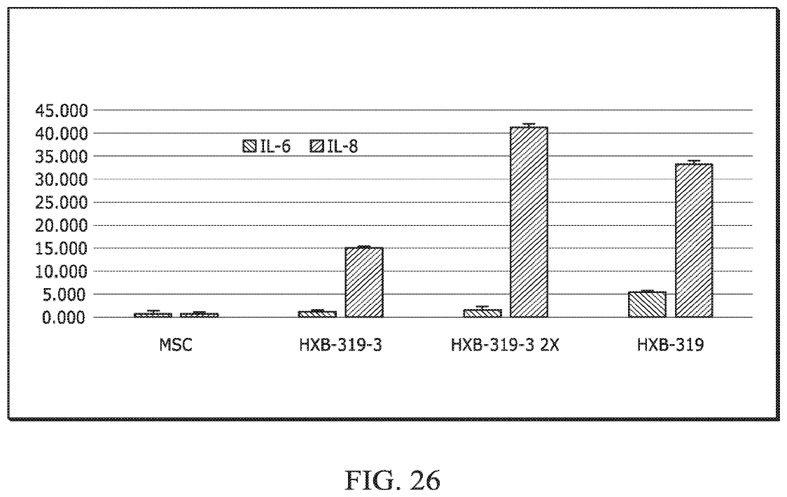

After it was discovered that TNF-α+INF-γ+Poly I:C three media component (HXB-319-3) gives similar anti-inflammatory results and similar gene expression seen in HXB-319, the inventors performed further experiments. IL-6 and IL-8 pro-inflammatory cytokines were not elevated using MSCs treated with HXB-319-3. The inventors then confirmed the dose effect of HXB-319-3 in vitro. The inventors performed IDO assays and RT-PCR (FIGS. 24-26).

The present disclosure is further described by the claims and documents provided herein.

While the present disclosure has been particularly shown and described with references to preferred embodiments thereof, it will be understood by those skilled in the art that various changes in form and details may be made therein without departing from the scope of the present disclosure encompassed by the appended claims. All patents, publications, and references cited in the foregoing specification are herein incorporated by reference in their entirety.

1. A priming medium for creating an isolated population of stem cells having an anti-inflammatory phenotype from an unprimed population of stem cells, the priming medium comprising:
a serum-free medium;
a functional activator of a Type I interferon (IFN) pathway and a Type II IFN pathway; and
at least two pro-inflammatory cytokines;
wherein the functional activator and the at least two pro-inflammatory cytokines are present in an amount sufficient to promote induction of stem cells having an anti-inflammatory phenotype;
wherein the cells having an anti-inflammatory phenotype are marked by increased expression and/or secretion of one or more anti-inflammatory or immune modulatory mediators as compared to the unprimed population of stem cells.

2. The priming medium of claim 1, wherein the functional activator is a toll-like receptor (TLR) ligand selected from the group consisting of Poly (I:C), lipopolysaccharide (LPS), tumor necrosis factor-α (TNF-α) and interleukin-1β (IL-1β).

3. The priming medium of claim 1, wherein the at least two proinflammatory cytokines are selected from the group consisting of IFN-γ, TNF-α, IL-1β and IL-17A.

4. The priming medium of claim 1, wherein the stem cells are mesenchymal stem cells (MSCs) or multipotent stromal cells.

5. The priming medium of claim 4, wherein the priming medium induces the MSCs to become pericyte-like MSCs that gain surface expression of CD146⁺ and Lep-R⁺.

6-8. (canceled)

9. The priming medium of claim 1, wherein the priming medium is a defined medium.

10-14. (canceled)

15. A priming medium for creating an isolated population of stem cells having an anti-inflammatory phenotype from an unprimed population of stem cells, the priming medium comprising:
a serum-free medium;
a functional activator of a Type I interferon (IFN) pathway and a Type II IFN pathway;
at least four pro-inflammatory cytokines; and
an essential vitamin;
wherein the functional activator and the at least four pro-inflammatory cytokines are present in an amount sufficient to promote induction of stem cells having an anti-inflammatory phenotype;
wherein the cells having an anti-inflammatory phenotype are marked by increased expression and/or secretion of one or more anti-inflammatory or immune modulatory mediators as compared to the unprimed population of stem cells.

16. The priming medium of claim 15, wherein the functional activator is a toll-like receptor (TLR) ligand selected from the group consisting of Poly (I:C), lipopolysaccharide (LPS), tumor necrosis factor-α (TNF-α) and interleukin-1β (IL-1β).

17. The priming medium of claim 15, wherein the at least four proinflammatory are IFN-γ, TNF-α, IL-1β and IL-17A.

18. The priming medium of claim 15, wherein the stem cells are mesenchymal stem cells (MSCs).

19. The priming medium of claim 18, wherein the priming medium induces the MSCs to become pericyte-like MSCs and gain surface expression of CD146⁺ and Lep-R⁺.

20. (canceled)

21. (canceled)

22. (canceled)

23. The priming medium of claim 15, wherein the priming medium is a defined medium.

24. The priming medium of claim 15, wherein the cells having an anti-inflammatory phenotype are marked by increased expression of indoleamine 2,3-deoxygenase (IDO), CD274, CD146 and platelet-derived growth factor receptor beta (PDGFRB), and by decreased expression of C-X3-C motif chemokine ligand 1 (CX3CL1), as compared to unprimed stem cells.

25. The priming medium of claim 24, wherein IDO expression is about 50,000 to about 150,000 times greater than IDO expression as compared to unprimed stem cells.

26. The priming medium of claim 15, wherein the cells having an anti-inflammatory phenotype upregulate production of cytotoxic T cells by at least 6-fold as compared to unprimed stem cells.

27. The priming medium of claim 15, wherein the cells having an anti-inflammatory phenotype upregulate production of Th1 cells by at least 12-fold as compared to unprimed stem cells.

28. The priming medium of claim 15, wherein the cells having an anti-inflammatory phenotype upregulate production of CD4+ T cells by at least 12-fold as compared to unprimed stem cells.

29. The priming medium of claim 15, wherein the cells having an anti-inflammatory phenotype upregulate production of natural killer (NK) cells greater than unprimed stem cells.

30. The priming medium of claim 15, wherein the cells having an anti-inflammatory phenotype suppress production of Th17 cells greater than unprimed stem cells.

31. The priming medium of claim 15, wherein the cells having an anti-inflammatory phenotype suppress production of double-stranded DNA autoantibodies as compared to unprimed stem cells.

32. The priming medium of claim 15, wherein the cells having an anti-inflammatory phenotype suppress production of B-cell activating factor (BAFF) as compared to unprimed stem cells.

33. The priming medium of claim 15, wherein the cells having an anti-inflammatory phenotype decrease production of IL-17 as compared to unprimed stem cells.

34-44. (canceled)
